(19) United States
(12) Patent Application Publication
(10) Pub. No.: US 2022/0296692 A1
(43) Pub. Date: Sep. 22, 2022

(54) USE OF AN ACTIVATABLE ANTI-PDL1 ANTIBODY AND AN ANTI-CTLA-4 ANTIBODY IN A NEOADJUVANT COMBINATION THERAPY FOR THE TREATMENT OF CANCER

(71) Applicant: CYTOMX THERAPEUTICS, INC., South San Francisco, CA (US)

(72) Inventors: Rachel Humphrey, South San Francisco, CA (US); Matthias Will, South San Francisco, CA (US); Yifah Yaron, South San Francisco, CA (US)

(21) Appl. No.: 17/618,658

(22) PCT Filed: Jun. 12, 2020
(86) PCT No.: PCT/US2020/037556

§ 371 Date: Dec. 13, 2021

Related U.S. Application Data
(60) Provisional application No. 62/860,953, filed on Jun. 13, 2019.

(51) Int. Cl. A61K 39/00; A61P 35/00; C07K 16/28

(52) U.S. Cl. CPC A61K 39/001129; A61P 35/00; C07K 16/2827; A61K 2039/507; A61K 2039/545; A61K 2039/876

(57) ABSTRACT

The invention relates generally to use of a neoadjuvant combination therapy of an activatable anti-PDL1 antibody and an anti-CTLA-4 antibody for the treatment of cancer.

### CROSS-REFERENCE TO RELATED APPLICATIONS

This application claims the benefit of U.S. Provisional Patent Application Ser. No. 62/860,953, filed on Jun. 13, 2019, which is incorporated by reference herein in its entirety.

### REFERENCE TO SEQUENCE LISTING

The “Sequence Listing” submitted electronically concurrently herewith pursuant to 37 C.F.R. § 1.821 in computer readable form (CFR) via EFS-Web as file name “sequencelisting.txt” is incorporated herein by reference. The electronic copy of the Sequence Listing was created on Jun. 12, 2020, and the disk size is 37.6 kilobytes.

### FIELD OF THE INVENTION

This invention generally relates to the use of an activatable anti-PDL1 antibody and an anti-CTLA-4 antibody in a neoadjuvant combination therapy for the treatment of cancer.

### BACKGROUND OF THE INVENTION

Antibody-based therapies have provided effective treatments for several diseases but in some cases, toxicities due to broad target expression have limited their therapeutic effectiveness. In addition, antibody-based therapeutics have exhibited other limitations such as rapid clearance from the circulation following administration.

In the realm of small molecule therapeutics, strategies have been developed to provide prodrugs of an active chemical entity. Such prodrugs are administered in a relatively inactive (or significantly less active) form. Once administered, the prodrug is metabolized in vivo into the active compound. Such prodrug strategies can provide for increased selectivity of the drug for its intended target and for a reduction of adverse effects.

Accordingly, there is a continued need in the field of antibody-based therapeutics for antibodies that mimic the desirable characteristics of the small molecule prodrug.

### SUMMARY OF THE INVENTION

In one aspect, the present invention provides a method of treating, alleviating a symptom of, or delaying the progression of a cancer in a subject having a solid tumor, the method comprising:

administering to the subject a neoadjuvant combination therapy comprising

(A) an activatable anti-PDL1 antibody, wherein the activatable anti-PDL1 antibody comprises:

(i) an antibody or antigen-binding portion thereof that binds human PDL1 (AB) that comprises:

a heavy chain variable region (VH) comprising a complementarity determining region 1 (CDRH1) that comprises the amino acid sequence of SEQ ID NO:125, a complementarity determining region 2 (CDRH2) that comprises the amino acid sequence of SEQ ID NO:126, and a complementarity determining region 3 (CDRH3) that comprises the amino acid sequence of SEQ ID NO:127, and

a light chain variable region (VL) comprising a light chain complementarity determining region 1 (CDRL1) that comprises the amino acid sequence of SEQ ID NO:128, a light chain complementarity determining region 2 (CDRL2) that comprises the amino acid sequence of SEQ ID NO:129, and a light chain complementarity determining region 3 (CDRL3) that comprises the amino acid sequence of SEQ ID NO:130;

(ii) a cleavable moiety (CM) linked, directly or indirectly, to the AB, wherein the CM is a polypeptide that functions as a substrate for a protease; and

(iii) a masking moiety (MM) linked, directly or indirectly, to the CM; and

(B) an anti-CTLA-4 antibody.

In another aspect, the present invention provides a method of treating, alleviating a symptom of, or delaying the progression of a cancer in a subject having a solid tumor, the method comprising administering to the subject a neoadjuvant combination therapy comprising

(A) an activatable anti-PDL1 antibody at a fixed dose of 800 mg, wherein the activatable anti-PDL1 antibody comprises:

(i) an antibody or antigen-binding portion thereof that binds human PDL1 (AB) that comprises:

a heavy chain variable region (VH) comprising a complementarity determining region 1 (CDRH1) that comprises the amino acid sequence of SEQ ID NO:125, a complementarity determining region 2 (CDRH2) that comprises the amino acid sequence of SEQ ID NO:126, and a complementarity determining region 3 (CDRH3) that comprises the amino acid sequence of SEQ ID NO:127, and

a light chain variable region (VL) comprising a light chain complementarity determining region 1 (CDRL1) that comprises the amino acid sequence of SEQ ID NO:128, a light chain complementarity determining region 2 (CDRL2) that comprises the amino acid sequence of SEQ ID NO:129, and a light chain complementarity determining region 3 (CDRL3) that comprises the amino acid sequence of SEQ ID NO:130;

(ii) a cleavable moiety (CM) linked, directly or indirectly, to the AB, wherein the CM is a polypeptide that functions as a substrate for a protease; and

(iii) a masking moiety (MM) linked, directly or indirectly, to the CM; and

(B) an anti-CTLA-4 antibody at a dose of 1 mg/kg.

In a further aspect, the present invention provides a method of treating, alleviating a symptom of, or delaying the progression of a cancer in a subject having a solid tumor, the method comprising administering to the subject a neoadjuvant combination therapy comprising

(A) an activatable anti-PDL1 antibody at a fixed dose of 800 mg,

wherein the activatable anti-PDL1 antibody comprises a heavy chain comprising the amino acid sequence of SEQ ID NO:122, and a light chain comprising an amino acid sequence selected from the group consisting of SEQ ID NO:123 and SEQ ID NO:124,

wherein the activatable anti-PDL1 antibody comprises an antibody or antigen-binding portion thereof that binds human PDL1 (AB), a cleavable moiety (CM), and a masking moiety (MM); and

(B) an anti-CTLA-4 antibody at a dose of 1 mg/kg. In some embodiments, the light chain comprises the amino acid sequence of SEQ ID NO:123. In other embodiments, the light chain comprises the amino acid sequence of SEQ ID NO:124.

In a still further aspect, the present invention provides an activatable anti-PDL1 antibody for use in the treatment of a cancer, wherein the treatment comprises administering the activatable anti-PDL1 antibody intravenously to a subject in a neoadjuvant combination with an anti-CTLA-4 antibody that is administered intravenously to the subject,

wherein the activatable anti-PDL1 antibody comprises:

(i) an antibody or antigen-binding portion thereof that binds human PDL1 (AB) that comprises:

a heavy chain variable region (VH) comprising a complementarity determining region 1 (CDRH1) that comprises the amino acid sequence of SEQ ID NO:125, a complementarity determining region 2 (CDRH2) that comprises the amino acid sequence of SEQ ID NO:126, and a complementarity determining region 3 (CDRH3) that comprises the amino acid sequence of SEQ ID NO:127, and

a light chain variable region (VL) comprising a light chain complementarity determining region 1 (CDRL1) that comprises the amino acid sequence of SEQ ID NO:128, a light chain complementarity determining region 2 (CDRL2) that comprises the amino acid sequence of SEQ ID NO:129, and a light chain complementarity determining region 3 (CDRL3) that comprises the amino acid sequence of SEQ ID NO:130;

(ii) a cleavable moiety (CM) linked, directly or indirectly, to the AB, wherein the CM is a polypeptide that functions as a substrate for a protease; and

(iii) a masking moiety (MM) linked, directly or indirectly, to the CM; and

(B) an anti-CTLA-4 antibody,

wherein the subject has a solid tumor.

In certain aspects, the cancer is a melanoma. In some embodiments, the cancer is resectable Stage III melanoma.

### BRIEF DESCRIPTION OF THE FIGURE

FIG. 1 provides a schematic representation of the neoadjuvant combination therapy study described in Example 1. In the study, a neoadjuvant combination therapy of an activatable anti-PDL1 antibody and ipilimumab is administered at a fixed dose of 800 mg and 3 mg/kg, respectively, every 3 weeks (q3w) for 2 infusions (Days 1-42), followed by surgical resection of the tumor on day 43. Six weeks after surgical resection of the tumor (beginning on day 85), a post-surgery combination therapy of the activatable anti-PDL1 antibody and the ipilimumab administered at a fixed dose of 800 mg and 1 mg/kg, respectively, every 3 weeks (q3w) for 2 infusions (Days 85-126). Beginning on day 127, an optional activatable anti-PDL1 antibody monotherapy is administered at a fixed dose of 800 mg every 2 weeks for up to one year.

### DETAILED DESCRIPTION

The present invention provides methods of treating, alleviating a symptom of, and/or delaying the progression of a cancer in a subject having a solid tumor by administering to the subject a neoadjuvant combination therapy that comprises an activatable anti-PDL1 antibody and an anti-CTLA-4 antibody. The subject is a mammal and typically, a human.

In one embodiment, the present invention provides a method of treating, alleviating a symptom of, and/or delaying the progression of a cancer in a subject having a solid tumor, the method comprising:

administering to the subject a neoadjuvant combination therapy comprising

(A) an activatable anti-PDL1 antibody, wherein the activatable anti-PDL1 antibody comprises:

(i) an antibody or antigen-binding portion thereof that binds human PDL1 (AB) that comprises:

a heavy chain variable region (VH) comprising a complementarity determining region 1 (CDRH1) that comprises the amino acid sequence of SEQ ID NO:125, a complementarity determining region 2 (CDRH2) that comprises the amino acid sequence of SEQ ID NO:126, and a complementarity determining region 3 (CDRH3) that comprises the amino acid sequence of SEQ ID NO:127, and

a light chain variable region (VL) comprising a light chain complementarity determining region 1 (CDRL1) that comprises the amino acid sequence of SEQ ID NO:128, a light chain complementarity determining region 2 (CDRL2) that comprises the amino acid sequence of SEQ ID NO:129, and a light chain complementarity determining region 3 (CDRL3) that comprises the amino acid sequence of SEQ ID NO:130;

(ii) a cleavable moiety (CM) linked, directly or indirectly, to the AB, wherein the CM is a polypeptide that functions as a substrate for a protease; and

(iii) a masking moiety (MM) linked, directly or indirectly, to the CM; and

(B) an anti-CTLA-4 antibody.

As used herein, the term “neoadjuvant” when used in connection with the term “combination therapy” refers to a combination therapy that is administered to a subject having a solid tumor prior to the subject undergoing surgical resection of all or a portion of the solid tumor. Thus, the neoadjuvant combination therapy is intended to enhance the outcome of the surgery procedure. In some embodiments, administering a neoadjuvant combination therapy to a subject prior to surgical resection of all or a portion of the solid tumor shrinks the tumor, such that the surgery is more effective and/or easier to perform. In some embodiments, administering a neoadjuvant combination therapy to a subject can result in the solid tumor being treatable via surgical resection, where the tumor would not be treatable via surgical resection in the absence of the neoadjuvant combination therapy. In some embodiments, administering a neoadjuvant combination therapy to a subject prior to surgical resection of all or a portion of the solid tumor results in fewer cancer cells remaining in the subject post-surgery (e.g., as compared to the number of cancer cells that would have remained in the subject in the absence of the neoadjuvant combination therapy). In some embodiments, administering a neoadjuvant combination therapy to a subject prior to surgical resection of all or a portion of the solid tumor results in no cancer cells remaining in the subject post-surgery (e.g., no cancer cells where the tumor used to be). In some embodiments, which are described in more detail hereinbelow, a post-surgical combination therapy comprising an activatable anti-PDL1 antibody and an anti-CTLA-4 antibody, and optionally a further post-surgical monotherapy comprising an activatable anti-PDL1 antibody is also administered to the subject. The term “post-surgical” when used in connection with the term “combination therapy” or “monotherapy” refers to a combination therapy or monotherapy that is administered to the subject at a time point after the procedure of surgical resection of all or a portion of the solid tumor. In some embodiments, a subject is administered a neoadjuvant combination therapy (e.g., an activatable anti-PDL1 antibody and an anti-CTLA-4 antibody) prior to surgical resection of all or a portion of a solid tumor, after which a post-surgical combination therapy comprising an activatable anti-PDL1 antibody and an anti-CTLA-4 antibody is administered to the subject. In some embodiments, a subject is administered a neoadjuvant combination therapy (e.g., an activatable anti-PDL1 antibody and an anti-CTLA-4 antibody) prior to surgical resection of all or a portion of a solid tumor, after which a post-surgical combination therapy (e.g., an activatable anti-PDL1 antibody and an anti-CTLA-4 antibody) is administered to the subject, and a further post-surgical monotherapy comprising the an activatable anti-PDL1 antibody is also administered to the subject after the post-surgical administration of the post-surgical combination therapy.

As used herein, the term “activatable anti-PDL1 antibody” refers to a compound comprising the following structure: an anti-PDL1 antibody or antigen binding portion thereof that binds human PDL1 (collectively, an “AB”) which is coupled, either directly or indirectly, to a prodomain that comprises a masking moiety (MM) and a cleavable moiety (CM). As used herein, the terms “human PDL1” and “PDL1” are used interchangeably herein to refer to human programmed death-ligand 1. The term “PD1” as used herein refers to human programmed cell death protein 1. In some embodiments, an activatable anti-PDL1 antibody comprises an anti-PDL1 antibody or antigen binding portion thereof that binds to non-human PDL1 (e.g., a mouse PDL1, a rat PDL1, a primate PDL1, a dog PDL1, a cat PDL1, a horse PDL1, a cow PDL1, a pig PDL1, or a sheep PDL1). It will be understood by those of ordinary skill in the art that embodiments described herein describing the properties, functions, or advantages of an activatable anti-PDL1 antibody that binds to human PDL1 will also be generally applicable to embodiments of activatable antibodies that bind to non-human PDL1. For examples, an “activatable anti-mouse PDL1 antibody” can be activated (e.g., unmasked) such that it is capable of binding to mouse PDL1.

The terms “masking moiety” and “MM”, are used interchangeably herein to refer to a peptide that, when positioned proximal to the AB, interferes with binding of the AB (and thus, the activatable anti-PDL1 antibody) to PDL1. The terms “cleavable moiety” and “CM” are used interchangeably herein to refer to a peptide that comprises a substrate for at least one protease (e.g., an endogenous protease that is present in the tumor microenvironment). The CM is positioned relative to the MM and AB components such that cleavage of the CM allows the release of the MM from its position proximal to the AB (also referred to herein as “unmasking” or “activation”). Thus, unmasking of the AB typically results in generation of an “activated” anti-PDL1 antibody that is capable of binding PDL1. The terms “uncleaved” or “intact” are used interchangeably herein to refer to an activatable anti-PDL1 antibody in which the prodomain portion is intact within the structure of the activatable anti-PDL1 antibody. The terms “peptide”, “polypeptide”, and “protein” are used interchangeably herein to refer to a polymer comprising naturally occurring or non-naturally occurring amino acid residues or amino acid analogues.

The AB component of the activatable anti-PDL1 antibody typically comprises at least a portion of the antigen binding domain of an anti-PDL1 antibody that has binding specificity for PDL1. As such, the activated anti-PDL1 antibody has specificity for PDL1. The term “antigen binding domain” refers herein to the part of the immunoglobulin molecule that participates in antigen binding. The antigen binding site is formed by amino acid residues of the variable (“V”) regions of the heavy (“H”) and light (“L”) chains. Three highly divergent stretches within the V regions of the heavy and light chains, referred to as “hypervariable regions”, are interposed between more conserved flanking stretches known as “framework regions” or “FRs”. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of an antigen, and the three hypervariable regions of each of the heavy and light chains are referred to as “complementarity-determining regions” or “CDRs”. Each of the heavy variable region (VH) and light chain variable region (VL) in the heavy and light chains, respectively, comprises three CDRs (CDR1, CDR2, and CDR3). The assignment of amino acids to each domain is in accordance with the definitions of Kabat Sequences of Proteins of Immunological Interest (National Institutes of Health, Bethesda, Md. (1987 and 1991); Chothia & Lesk, J. Mol. Biol. 196:901-917 (1987); Chothia, et al. Nature 342:878-883 (1989), which are incorporated herein by reference in their entireties).

In a specific embodiment, the AB component of the activatable anti-PDL1 antibody comprises a light chain variable region (VL) comprising variable light chain CDRs that comprise the amino acid sequences corresponding to SEQ ID NOs:128 (CDRL1), 129 (CDRL2), and SEQ ID NO:130 (CDRL3) and a heavy chain variable region (VH) comprising variable heavy chain CDRs that comprise the amino acid sequences corresponding to SEQ ID NOs:125 (CDRH1), SEQ ID NO:126 (CDRH2), and SEQ ID NO:127 (CDRH3). ABs having this specific set of CDR sequences have been demonstrated to have binding specificity for human PDL1, as described in PCT Publ. Nos. WO 2016/149201 and WO 2018/222949, each of which is incorporated herein by reference.

Activatable anti-PDL1 antibodies employed in the practice of the present invention may have an AB that comprises, for example, one or more light chain variable region (VL), heavy chain variable region (VH), or hypervariable region of a light and/or heavy chain, variable fragment (Fv), Fab′ fragment, F(ab′)2 fragments, Fab fragment, single chain antibody (scab), single chain variable region (scFv), complementarity determining region (CDR), domain antibody (dAB), single domain heavy chain immunoglobulin of the BHH or BNAR type, single domain light chain immunoglobulins, or other polypeptide known to bind human PDL1. In some embodiments, the AB comprises an immunoglobulin comprising two Fab regions and an Fc region. In certain embodiments, the activatable anti-PDL1 antibody is multivalent, e.g., bivalent, trivalent, and so on. Often, the activatable anti-PDL1 antibody comprises two light chains (each comprising a VL region) and two heavy chains (each comprising a VH region). In certain embodiments, each light chain comprises a prodomain linked directly or indirectly (e.g., via a linker) to the VL. In some of these embodiments, the two light chains are identical to each other with respect to amino acid sequence and similarly, the two heavy chains are identical to each other with respect to amino acid sequence. In some of these embodiments, the two light chains are identical to each other with respect to amino acid sequence, while the two heavy chains are not identical to each other with respect to amino acid sequence. In some of these embodiments, the two light chains are not identical to each other with respect to amino acid sequence, while the two heavy chains are identical to each other with respect to amino acid sequence. In some of these embodiments, the two light chains are not identical to each other with respect to amino acid sequence and the two heavy chains are not identical to each other with respect to amino acid sequence. In some of these embodiments, the two light chains differ from each other by one or more (e.g., 1, 2, 3, 4, 5, 6, 7, 8, 9, 10, 15, 20, 25, 30, or more) amino acid residues and/or the two heavy chains differ from each other by one or more (e.g., 1, 2, 3, 4, 5, 6, 7, 8, 9, 10, 15, 20, 25, 30, or more) amino acid residues. In some of these embodiments, the two light chains differ from each other by one or more (e.g., 1, 2, 3, 4, 5, 6, 7, 8, 9, 10, 15, 20, 25, 30, or more) amino acid residues while having identical CDR sequences and/or the two heavy chains differ from each other by one or more (e.g., 1, 2, 3, 4, 5, 6, 7, 8, 9, 10, 15, 20, 25, 30, or more) amino acid residues while having identical CDR sequences. In some of these embodiments, the amino acid sequences of the two light chains are at least 80% identical to each other (e.g., at least 80%, at least 85%, at least 90%, at least 95%, at least 96%, at least 97%, at least 98%, at least 99% identical) and/or the amino acid sequences of the two heavy chains are at least 80% identical to each other (e.g., at least 80%, at least 85%, at least 90%, at least 95%, at least 96%, at least 97%, at least 98%, at least 99% identical). In some of these embodiments, the amino acid sequences of the two light chains are at least 80% identical to each other (e.g., at least 80%, at least 85%, at least 90%, at least 95%, at least 96%, at least 97%, at least 98%, at least 99% identical) while having identical CDR sequences and/or the amino acid sequences of the two heavy chains are at least 80% identical to each other (e.g., at least 80%, at least 85%, at least 90%, at least 95%, at least 96%, at least 97%, at least 98%, at least 99% identical) while having identical CDR sequences.

The presence of the prodomain in the activatable anti-PDL1 antibody thus confers the potential for reduced toxicity and/or adverse side effects that may otherwise result from binding of the AB at non-treatment sites if the AB were not masked or otherwise inhibited from binding to the PDL1 target.

Masking moieties suitable for use in the practice of the present invention include those which, when employed in the structure of an activatable anti-PDL1 antibody, function to reduce the binding affinity of the activatable anti-PDL1 antibody to human PDL1, as compared to the binding affinity of the corresponding parental anti-PDL1 AB to human PDL1. As used herein, the term “parental AB” refers to the AB without a prodomain. In some embodiments, the MM is selected such that the binding affinity of the activatable anti-PDL1 antibody to human PDL1 is reduced by at least 50%, 60%, 70%, 80%, 90%, 92%, 93%, 94%, 95%, 96%, 97%, 98%, 99% and even 100% for at least 2, 4, 6, 8, 12, 28, 24, 30, 36, 48, 60, 72, 84, or 96 hours, or 5, 10, 15, 30, 45, 60, 90, 120, 150, or 180 days, or 1, 2, 3, 4, 5, 6, 7, 8, 9, 10, 11, or 12 months or more, relative to the binding of the corresponding parental AB to human PDL1, when measured in vivo or in an in vitro assay, such as those described in PCT Publication No. WO 2016/149201, which is incorporated herein by reference in its entirety.

In some embodiments, an MM is selected such that the resulting activatable anti-PDL1 antibody exhibits a binding affinity to human PDL1 that is at least 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1000-10,000,000, 10,000-100,000, 10,000-1,000,000, 10,000-10,000,000, 100,000-1,000,000, or 100,000-10,000,000 times lower than the binding affinity of the corresponding parental AB to human PDL 1. As used herein, all numerical ranges set forth hereinabove and hereinbelow are intended to be inclusive of the numerical limits that define the range.

Masking moieties that are suitable for use in the activatable anti-PDL1 antibodies employed herein can be readily identified using any of a variety of known techniques, including those described in PCT Publication No. WO 2009/025846, which is hereby incorporated by reference in its entirety.

Often, the MM is a polypeptide of about 2 to 40 amino acids in length. In some embodiments, the MM is a polypeptide of up to about 40 amino acids in length. In certain embodiments, the amino acid sequence of the MM polypeptide is different from that of the amino acid sequence of the target human PDL1. In some embodiments, the MM polypeptide sequence is no more than 50% identical to any human PDL1 amino acid sequence. In some embodiments, the MM polypeptide sequence is no more than 40%, 30%, 25%, 20%, 15%, or 10% identical to the amino acid sequence of the target PDL1. The percent identity of two sequences is determined by optimal alignment of the test polypeptide sequence with a reference polypeptide sequence using a program such as GAP or BESTFIT using default gap weights.

Exemplary masking moieties include those which comprise any one of the following amino acid sequences: YCEVSELFVLPWCMG (SEQ ID NO:1), SCLMHPHYAHDYCYV (SEQ ID NO:2), LCEVLMLLQHPWCMG (SEQ ID NO:3), IACRHFMEQLPFCHH (SEQ ID NO:4), FGPRCGEASTCVPYE (SEQ ID NO:5), ILYCDSWGAGCLTRP (SEQ ID NO:6), GIALCPSHFCQLPQT (SEQ ID NO:7), DGPRCFVSGECSPIG (SEQ ID NO:8), LCYKLDYDDRSYCHI (SEQ ID NO:9), PCHPHPYDARPYCNV (SEQ ID NO:10), PCYWHPFFAYRYCNT (SEQ ID NO:11), VCYYMDWLGRNWCSS (SEQ ID NO:12), LCDLFKLREFPYCMG (SEQ ID NO:13), YLPCHFVPIGACNNK (SEQ ID NO:14), IFCHMGVVVPQCANY (SEQ ID NO:15), ACHPHPYDARPYCNV (SEQ ID NO:16), PCHPAPYDARPYCNV (SEQ ID NO:17), PCHPHAYDARPYCNV (SEQ ID NO:18), PCHPHPADARPYCNV (SEQ ID NO:19), PCHPHPYAARPYCNV (SEQ ID NO:20), PCHPHPYDAAPYCNV (SEQ ID NO:21), PCHPHPYDARPACNV (SEQ ID NO:22), PCHPHPYDARPYCAV (SEQ ID NO:23), PCHAHPYDARPYCNV (SEQ ID NO:24), and PCHPHPYDARAYCNV (SEQ ID NO:25). Often, the activatable anti-PDL1 antibody comprises an MM that comprises the amino acid sequence GIALCPSHFCQLPQT (SEQ ID NO:7).

In some embodiments, the MM comprises an amino acid sequence that is at least 90%, 91%, 92%, 93%, 94%, 95%, 96%, 97%, 98% or 99% identical to an amino acid sequence selected from the group consisting of SEQ ID NOs:1-25.

Suitable substrates for use in the CM may be identified using any of a variety of known techniques include those described in U.S. Pat. Nos. 7,666,817, 8,563,269, PCT Publication No. WO 2014/026136, and Boulware et al. “Evolutionary optimization of peptide substrates for proteases that exhibit rapid hydrolysis kinetics,” Biotechnol Bioeng. 106.3 (2010): 339-46, each of which is incorporated by reference in their entireties.

In some embodiments, the protease that cleaves the CM is active, e.g., up-regulated in diseased tissue and the protease cleaves the CM in the activatable antibody when the activatable antibody is exposed to the protease. Typically, the disease tissue is tumor tissue. In some embodiments, the protease is co-localized with PDL1 in a tissue, and the protease cleaves the CM in the activatable antibody when the activatable antibody is exposed to the protease. In some embodiments, the protease is present at relatively higher levels in or in close proximity to PDL1 target-containing tissue of a treatment site or diagnostic site than in tissue of non-treatment sites (for example in healthy tissue), and the protease cleaves the CM in the activatable antibody when the activatable antibody is exposed to the protease.

Illustrative CMs that are suitable for use in the activatable anti-PDL1 antibodies employed herein include those comprising any one of the following amino acid sequences: LSGRSDNH (SEQ ID NO:26), TGRGPSWV (SEQ ID NO:27), PLTGRSGG (SEQ ID NO:28), TARGPSFK (SEQ ID NO:29), NTLSGRSENHSG (SEQ ID NO:30), NTLSGRSGNHGS (SEQ ID NO:31), TSTSGRSANPRG (SEQ ID NO:32) TSGRSANP, (SEQ ID NO:33), VHMPLGFLGP (SEQ ID NO:34), AVGLLAPP (SEQ ID NO:35), AQNLLGMV (SEQ ID NO: 36), QNQALRMA (SEQ ID NO:37), LAAPLGLL (SEQ ID NO:38), STFPFGMF (SEQ ID NO: 39), ISSGLLSS (SEQ ID NO:40), PAGLWLDP (SEQ ID NO:41), VAGRSMRP (SEQ ID NO: 42), VVPEGRRS (SEQ ID NO:43), ILPRSPAF (SEQ ID NO:44), MVLGRSLL (SEQ ID NO: 45), QGRAITFI (SEQ ID NO:46), SPRSIMLA (SEQ ID NO:47), SMLRSMPL (SEQ ID NO: 48), ISSGLLSGRSDNH (SEQ ID NO:49), AVGLLAPPGGLSGRSDNH (SEQ ID NO:50), ISSGLLSSGGSGGSLSGRSDNH (SEQ ID NO:51), LSGRSGNH (SEQ ID NO:52), SGRSANPRG (SEQ ID NO:53), LSGRSDDH (SEQ ID NO:54), LSGRSDIH (SEQ ID NO:55), LSGRSDQH (SEQ ID NO:56), LSGRSDTH (SEQ ID NO:57), LSGRSDYH (SEQ ID NO:58), LSGRSDNP (SEQ ID NO:59), LSGRSANP (SEQ ID NO:60), LSGRSANI (SEQ ID NO:61), LSGRSDNI (SEQ ID NO:62), MIAPVAYR (SEQ ID NO:63), RPSPMWAY (SEQ ID NO:64), WATPRPMR (SEQ ID NO:65), FRLLDWQW (SEQ ID NO:66), ISSGL (SEQ ID NO:67), ISSGLLS (SEQ ID NO:68), ISSGLL (SEQ ID NO:69), ISSGLLSGRSANPRG (SEQ ID NO: 70), AVGLLAPPTSGRSANPRG (SEQ ID NO:71), AVGLLAPPSGRSANPRG (SEQ ID NO:72), ISSGLLSGRSDDH (SEQ ID NO:73), ISSGLLSGRSDIH (SEQ ID NO:74), ISSGLLSGRSDQH (SEQ ID NO:75) ISSGLLSGRSDTH (SEQ ID NO:76), ISSGLLSGRSDYH (SEQ ID NO:77), ISSGLLSGRSDNP (SEQ ID NO:78), ISSGLLSGRSANP (SEQ ID NO:79) ISSGLLSGRSANI (SEQ ID NO:80), AVGLLAPPGGLSGRSDDH (SEQ ID NO:81), AVGLLAPPGGLSGRSDIH (SEQ ID NO:82), AVGLLAPPGGLSGRSDQH (SEQ ID NO: 83), AVGLLAPPGGLSGRSDTH (SEQ ID NO: 84), AVGLLAPPGGLSGRSDYH (SEQ ID NO: 85), AVGLLAPPGGLSGRSDNP (SEQ ID NO:86), AVGLLAPPGGLSGRSANP (SEQ ID NO: 87), AVGLLAPPGGLSGRSANI (SEQ ID NO:88), ISSGLLSGRSDNI (SEQ ID NO:89), AVGLLAPPGGLSGRSDNI (SEQ ID NO:90), GLSGRSDNHGGAVGLLAPP (SEQ ID NO:91), and GLSGRSDNHGGVHMPLGFLGP (SEQ ID NO:92). In a specific embodiment, the activatable anti-PDL1 antibody comprises a CM having the amino acid sequence, ISSGLLSGRSDNH (SEQ ID NO:49).

Activatable anti-PDL1 antibodies employed in the practice of the present invention may exist in a variety of structural configurations. Exemplary formulae for activatable antibodies are provided below. It should be noted that although MM and CM are indicated as distinct components in the formulae below, in all exemplary embodiments (including formulae) disclosed herein it is contemplated that the amino acid sequences of the MM and the CM could overlap, e.g., such that the CM is completely or partially contained within the MM.

MM, CM, and AB components of the activatable anti-PDL1 antibody may be arranged as indicated in the following formulas (in order from N- to C-terminal):

(MM)-(CM)-(AB)

(AB)-(CM)-(MM)

where MM, CM, and AB are as previously defined, and where each “-” refers independently to a direct or indirect (i.e., via a linker as described hereinbelow) linkage.

In many embodiments, it may be desirable to insert one or more linkers into the activatable anti-PDL1 antibody construct to impart flexibility at one or more of the MM-CM junction, the CM-AB junction, or both. For example, in certain embodiments, the activatable anti-PDL1 antibody may comprise one of the following formulae (where the formula below represents an amino acid sequence in either N- to C-terminal direction or C- to N-terminal direction):

(MM)-L1-(CM)-(AB)

(MM)-(CM)-L2-(AB)

(MM)-L1-(CM)-L2-(AB)

wherein MM, CM, and AB are as defined hereinabove; wherein L1 and L2 may be the same or different, and each independently may be optionally present or absent.

Linkers suitable for use in the activatable anti-PDL1 antibodies employed in the practice of the present invention may be any of a variety of lengths. Suitable linkers include those having a length in the range of from about 1 to about 20 amino acids, or from about 1 to about 19 amino acids, or from about 1 to about 18 amino acids, or from about 1 to about 17 amino acids, or from about 1 to about 16 amino acids, or from about 1 to about 15 amino acids, or from about 2 to about 15 amino acids, or from about 3 to about 15 amino acids, or from about 3 to about 14 amino acids, or from about 3 to about 13 amino acids, or from about 3 to about 12 amino acids. In some embodiments, the activatable anti-PDL1 antibody comprises one or more linkers each independently comprising 1, 2, 3, 4, 5, 6, 7, 8, 9, 10, 11, 12, 13, 14, 15, 16, 17, 18, 19, or 20 amino acid residues.

Typically, the linker is a flexible linker comprising one or more amino acid residues selected from the group consisting of Gly, Ser, Ala, and Thr, and often, the linker comprises one or more amino acid residues selected from the group consisting of Gly and Ser. Exemplary flexible linkers include a glycine homopolymer (G)ₙ (wherein n is an integer that is at least 1, or an integer in the range of from about 1 to about 30, or an integer in the range of from about 1 to about 25, or an integer in the range of from about 1 to about 20, or an integer in the range of from about 1 to about 15, or an integer in the range of from about 1 to about 10); a glycine-serine co-polymer, including, for example, (GS)ₙ (wherein n is an integer that is at least 1, or an integer in the range of from about 1 to about 30, or an integer in the range of from about 1 to about 25, or an integer in the range of from about 1 to about 20, or an integer in the range of from about 1 to about 15, or an integer in the range of from about 1 to about 10), (GSGGS)ₙ (SEQ ID NO:93) (wherein n is an integer that is at least 1, or an integer in the range of from about 1 to about 30, or an integer in the range of from about 1 to about 25, or an integer in the range of from about 1 to about 20, or an integer in the range of from about 1 to about 15, or an integer in the range of from about 1 to about 10), (GGGS)ₙ (SEQ ID NO:94) (wherein n is an integer that is at least 1, or an integer in the range of from about 1 to about 30, or an integer in the range of from about 1 to about 25, or an integer in the range of from about 1 to about 20, or an integer in the range of from about 1 to about 15, or an integer in the range of from about 1 to about 10); a linker that comprises or consists of glycine and serine residues, such as, for example, GGSG (SEQ ID NO:95), GGSGG (SEQ ID NO:96), GSGSG (SEQ ID NO:97, GSGGG (SEQ ID NO:98), GSSGGSGGSGG (SEQ ID NO:99), GSSGGSGGSGGS (SEQ ID NO:100), GSSGGSGGSGGSGGGS (SEQ ID NO:101), GSSGGSGGSG (SEQ ID NO:102), GSSGGSGGSGS (SEQ ID NO:103), GGGS (SEQ ID NO:104); GSSG (SEQ ID NO:106), GGGSSGGSGGSGG (SEQ ID NO:107), GGS, and the like; a linker that comprises or consists of glycine, serine, and threonine residues, such as, for example, GSSGT (SEQ ID NO:105); a glycine-alanine co-polymer; an alanine-serine co-polymer; as well as other flexible linkers known in the art.

Activatable anti-PDL1 antibodies employed in the practice of the present invention may also comprise a spacer located, for example, at the amino terminus of the MM. In some embodiments, the spacer is joined directly to the MM of the activatable anti-PDL1 antibody, for example, in the structural arrangement, from N-terminus to C-terminus, of spacer-MM-CM-AB, wherein each “-” refers independently to a direct or indirect (i.e., via any of the linkers described herein) linkage. Illustrative spacer amino acid sequences may comprise or consist of any of the following exemplary amino acid sequences: QGQSGS (SEQ ID NO:108); GQSGS (SEQ ID NO:109); QSGS (SEQ ID NO:110); SGS; GS; S; QGQSGQG (SEQ ID NO:111); GQSGQG (SEQ ID NO:112); QSGQG (SEQ ID NO:113); SGQG (SEQ ID NO:114); GQG; QG; G; QGQSGQ (SEQ ID NO:115); GQSGQ (SEQ ID NO:116); QSGQ (SEQ ID NO:117); SGQ; GQ; and Q.

Thus in some embodiments, the spacer comprises or consists of the amino acid sequence QGQSGS (SEQ ID NO:108). In some embodiments, the spacer comprises or consists of the amino acid sequence GQSGS (SEQ ID NO:109). In some embodiments, the spacer comprises or consists of the amino acid sequence QSGS (SEQ ID NO:110). In some embodiments, the spacer comprises or consists of the amino acid sequence SGS. In some embodiments, the spacer comprises or consists of the amino acid sequence GS. In some embodiments, the spacer comprises or consists of the amino acid residue S. In some embodiments, the spacer comprises or consists of the amino acid sequence QGQSGQG (SEQ ID NO:111). In some embodiments, the spacer comprises or consists of the amino acid sequence GQSGQG (SEQ ID NO:112). In some embodiments, the spacer comprises or consists of the amino acid sequence QSGQG (SEQ ID NO:113). In some embodiments, the spacer comprises or consists of the amino acid sequence SGQG (SEQ ID NO:114). In some embodiments, the spacer comprises or consists of the amino acid sequence GQG. In some embodiments, the spacer comprises or consists of the amino acid sequence QG. In some embodiments, the spacer comprises or consists of the amino acid residue G. In some embodiments, the spacer comprises or consists of the amino acid sequence QGQSGQ (SEQ ID NO:115). In some embodiments, the spacer comprises or consists of the amino acid sequence GQSGQ (SEQ ID NO:116). In some embodiments, the spacer comprises or consists of the amino acid sequence QSGQ (SEQ ID NO:117). In some embodiments, the spacer comprises or consists of the amino acid sequence SGQ. In some embodiments, the spacer comprises or consists of the amino acid sequence GQ. In some embodiments, the spacer comprises or consists of the amino acid residue Q. In some embodiments, the activatable anti-PDL1 antibody does not include a spacer sequence.

In a specific embodiment, the activatable anti-PDL1 antibody is PL07-2001-C5H9v2, which comprises two light chains and two heavy chains. Each light chain comprises a prodomain amino acid sequence (i.e., the prodomain comprising an MM and a CM) positioned N-terminal to a VL amino acid sequence. The variable light chain (VL) amino acid sequence in each light chain of PL07-2001-C5H9v2 comprises the amino acid sequence of SEQ ID NO: 119:

(SEQ ID NO: 119)
DIQMTQSPSSLSASVGDRVTITCRASQSISSYLNWYQQKPGKAPKLLIY
AASSLQSGVPSRFSGSGSGTDFTLTISSLQPEDFATYYCQQDNGYPSTF
GGGTKVEIKR

CDRL1, CDRL2, and CDRL3 are each indicated by underscored text.

Each heavy chain of PL07-2001-C5H9v2 comprises a heavy chain variable region (VH) comprising the amino acid sequence of SEQ ID NO:118:

(SEQ ID NO: 118)
EVQLLESGGGLVQPGGSLRLSCAASGFTFSSYAMSWVRQAPGKGLEWVSS
IWRNGIVTVYADSVKGRFTISRDNSKNTLYLQMNSLRAEDTAVYYCAKWS
AAFDYWGQGTLVTVSS

CDRH1, CDRH2, and CDRH3 are indicated by underscored text.

Thus, in one embodiment, the activatable anti-PDL1 antibody employed in the practice of the present invention comprises a VL comprising the amino acid sequence of SEQ ID NO:119 and a VH comprising the amino acid sequence of SEQ ID NO:118. The VL and VH of the heavy and light chains together form the AB of the activatable anti-PDL1 antibody.

The amino acid sequence of each light chain of PL07-2001-C5H9v2, which includes a spacer, MM, CM, VL, and human kappa constant domain, is set forth in SEQ ID NO:124:

(SEQ ID NO: 124)
QGQSGSGIALCPSHFCQLPQTGGGSSGGSGGSGGISSGLLSGRSDNHGGS
DIQMTQSPSSLSASVGDRVTITCRASQSISSYLNWYQQKPGKAPKLLIYA
ASSLQSGVPSRFSGSGSGTDFTLTISSLQPEDFATYYCQQDNGYPSTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

The spacer sequence is indicated by underscored text (corresponding to SEQ ID NO:108), the MM sequence is indicated by italicized text (corresponding to SEQ ID NO:7), and the CM is indicated by bolded text (corresponding to SEQ ID NO:49). The VL sequence is indicated by underscored and italicized text (corresponding to SEQ ID NO:119). Between the C-terminus of the MM sequence and the N-terminus of the CM sequence is a first linker sequence (corresponding to SEQ ID NO:107). Between the C-terminus of the CM sequence and the N-terminus of the VL sequence is a second linker sequence, GGS.

Each heavy chain sequence of PL07-2001-C5H9v2 comprises the sequence of SEQ ID NO:122:

(SEQ ID NO: 122)
EVQLLESGGGLVQPGGSLRLSCAASGFTFSSYAMSWVRQAPGKGLEWVSS
IWRNGIVTVYADSVKGRFTISRDNSKNTLYLQMNSLRAEDTAVYYCAKWS
AAFDYWGQGTLVTVSSASTKGPSVFPLAPCSRSTSESTAALGCLVKDYFP
EPVTVSWNSGALTSGVHTFPAVLQSSGLYSLSSVVTVPSSSLGTKTYTCN
VDHKPSNTKVDKRVESKYGPPCPPCPAPEFLGGPSVFLFPPKPKDTLMIS
RTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSF
FLYSRLTVDKSRWQEGNVFSCSVMHEALHNHYTQKSLSLSLG

The heavy chain sequence of PL07-2001-C5H9v2 comprises the VH of SEQ ID NO:118 and the amino acid sequence of IgG4 S229P. Thus, in a specific embodiment, the methods of the present invention employ the activatable anti-PDL1 antibody comprising a light chain comprising the amino acid sequence of SEQ ID NO:124 and a heavy chain comprising the amino acid sequence of SEQ ID NO:122, wherein the light chain comprises an MM, a CM, and a VL (in which the MM and CM are positioned within a prodomain). The activatable anti-PDL1 antibody typically comprises two light chains and two heavy chains.

Activatable anti-PDL1 antibodies suitable for use in the practice of the invention may thus comprise a light chain comprising the MM-L1-CM-L2-VL structure of each light chain in PL07-2001-C5H9v2, embodied by the sequence corresponding to SEQ ID NO:120:

(SEQ ID NO: 120)
GIALCPSHFCQLPQTGGGSSGGSGGSGGISSGLLSGRSDNHGGSDIQMTQ
SPSSLSASVGDRVTITCRASQSISSYLNWYQQKPGKAPKLLIYAASSLQS
GVPSRFSGSGSGTDFTLTISSLQPEDFATYYCQQDNGYPSTFGGGTKVEI
KR.

In some of these embodiments, the light chain comprises the above-described MM-L1-CM-L2-VL-human kappa constant domain structure of PL07-2001-C5H9v2, as set forth in SEQ ID NO:123:

(SEQ ID NO: 123)
GIALCPSHFCQLPQTGGGSSGGSGGSGGISSGLLSGRSDNHGGSDIQMTQ
SPSSLSASVGDRVTITCRASQSISSYLNWYQQKPGKAPKLLIYAASSLQS
GVPSRFSGSGSGTDFTLTISSLQPEDFATYYCQQDNGYPSTFGGGTKVEI
KRTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVT
KSFNRGEC

In these embodiments, each heavy chain typically comprises a VH comprising the amino acid sequence of SEQ ID NO:118.

Similarly, suitable activatable anti-PDL1 antibodies may comprises the spacer-MM-L1-CM-L2 structure of each light chain in PL07-2001-C5H9v2, embodied by the sequence corresponding to SEQ ID NO:121:

(SEQ ID NO: 121)
QGQSGSGIALCPSHFCQLPQTGGGSSGGSGGSGGISSGLLSGRSDNHGGS
DIQMTQSPSSLSASVGDRVTITCRASQSISSYLNWYQQKPGKAPKLLIYA
ASSLQSGVPSRFSGSGSGTDFTLTISSLQPEDFATYYCQQDNGYPSTFGG
GTKVEIKR

In these embodiments, each heavy chain typically comprises a VH comprising the amino acid sequence of SEQ ID NO:118.

Activatable anti-PDL1 antibodies employed in the practice of the above-described methods may comprise any of the MM, CM, and AB components described herein. In a particular embodiment, the MM comprises the amino acid sequence of SEQ ID NO:7. In these and other embodiments, the CM comprises the amino acid sequence of SEQ ID NO:49. In some of these embodiments, the AB comprises a heavy chain variable region (VH) comprising the amino acid sequence of SEQ ID NO:118 and a light chain variable region (VL) comprising the amino acid sequence of SEQ ID NO:119.

In some embodiments, the activatable anti-PDL1 antibody comprises a light chain and a heavy chain, wherein the light chain comprises the MM, the CM, and VL, and wherein the light chain comprises the amino acid sequence of SEQ ID NO:120, and wherein the heavy chain comprises a VH comprising the amino acid sequence of SEQ ID NO:118. In another embodiment, the activatable anti-PDL1 antibody comprises a light chain and a heavy chain, wherein the light chain comprises a spacer, the MM, the CM, and VL, and wherein the light chain comprises the amino acid sequence of SEQ ID NO:121, and wherein the heavy chain comprises a VH comprising the amino acid sequence of SEQ ID NO:118.

In some embodiments, multiple doses of the neoadjuvant combination therapy (e.g., an activatable anti-PDL1 antibody and an anti-CTLA-4 antibody) are administered to the subject (e.g., prior to the subject undergoing surgical resection of all or a portion of the solid tumor). As used herein, reference to “dose” in connection with a combination therapy described herein is intended to mean a dose of each component of the neoadjuvant combination therapy. In some embodiments, two, three, four, five, six, seven, eight, nine, or ten or more doses of the neoadjuvant combination therapy may be administered to the subject. Typically, a dose of the neoadjuvant combination therapy is administered once every three weeks (21 days). In some embodiments, a dose of the neoadjuvant combination therapy is administered once every week. In some embodiments, a dose of the neoadjuvant combination therapy is administered once every two weeks. In some embodiments, a dose of the neoadjuvant combination therapy is administered once every four weeks. In some embodiments, a dose of the neoadjuvant combination therapy is administered once every 5, 6, 7, 8, 9, 10, 11, 12, 13, 14, 15, 16, 17, 18, 19, 20, 21, 22, 23, 24, 25, 26, 27, or 28 days. In some embodiments, doses of the neoadjuvant combination therapy are administered at a constant frequency (e.g., two or more doses of the neoadjuvant combination therapy can be administered once every 3 weeks). In some embodiments, doses of the neoadjuvant combination therapy are administered at a variable frequency (e.g., the time period between the first two doses of the neoadjuvant combination therapy can 3 weeks, and future doses of the neoadjuvant combination therapy can be administered weekly). As will be understood by a person of ordinary skill in the art, other constant and variable dosing periods for the neoadjuvant combination therapy described herein can be employed. In certain of these embodiments, two doses of the neoadjuvant combination therapy are administered to the subject, prior to surgical resection of all or a portion of the solid tumor.

In certain of these embodiments, the activatable anti-PDL1 antibody component of the neoadjuvant combination therapy is administered at a fixed dose in the range of from 240 mg to 2400 mg. In some embodiments, the activatable anti-PDL1 antibody component of the neoadjuvant combination therapy is administered at a fixed dose of 800 mg. In other embodiments, the activatable anti-PDL1 antibody component of the neoadjuvant combination therapy is administered to the subject at a dose in the range of from 0.3 mg/kg to 30 mg/kg. In certain embodiments, the activatable anti-PDL1 antibody component of the neoadjuvant combination therapy is administered to the subject at a dose of 0.3 mg/kg, 1.0 mg/kg, 3.0 mg/kg, 10.0 mg/kg or 30.0 mg/kg. In certain of these embodiments, the activatable anti-PDL1 antibody component of the neoadjuvant combination therapy is administered at a fixed dose in the range of from about 240 mg to about 2400 mg. In some embodiments, the activatable anti-PDL1 antibody component of the neoadjuvant combination therapy is administered at a fixed dose of about 800 mg. In other embodiments, the activatable anti-PDL1 antibody component of the neoadjuvant combination therapy is administered to the subject at a dose in the range of from about 0.3 mg/kg to about 30 mg/kg. In certain embodiments, the activatable anti-PDL1 antibody component of the neoadjuvant combination therapy is administered to the subject at a dose of about 0.3 mg/kg, about 1.0 mg/kg, about 3.0 mg/kg, about 10.0 mg/kg or about 30.0 mg/kg.

In some embodiments, the anti-CTLA-4 component of the neoadjuvant combination therapy is administered at a dose in the range of from 0.3 mg/kg to 30 mg/kg. Sometimes the anti-CTLA-4 component of the neoadjuvant combination therapy is administered at a dose in the range of from 0.1 mg/kg to 20 mg/kg, or in the range of from 0.1 mg/kg to 15 mg/kg, or in the range of from 0.1 mg/kg to 10 mg/kg, or in the range of from 0.5 mg/kg to 10 mg/kg, or in the range of from 0.5 mg/kg to 5 mg/kg, or in the range of from 0.5 mg/kg to 3 mg/kg, or in the range of from 0.5 mg/kg to 2 mg/kg. In certain embodiments, the anti-CTLA-4 antibody component of the neoadjuvant combination therapy is administered at a dose of less than 3 mg/kg, or less than 2 mg/kg. Sometimes, the anti-CTLA-4 component of the neoadjuvant combination therapy is administered at a dose selected from the group consisting of 1 mg/kg and 2 mg/kg. Often, the anti-CTLA-4 antibody component of the neoadjuvant combination therapy is administered at a dose of 1 mg/kg. In some embodiments, the anti-CTLA-4 component of the neoadjuvant combination therapy is administered at a dose in the range of from about 0.3 mg/kg to about 30 mg/kg. Sometimes the anti-CTLA-4 component of the neoadjuvant combination therapy is administered at a dose in the range of from about 0.1 mg/kg to about 20 mg/kg, or in the range of from about 0.1 mg/kg to about 15 mg/kg, or in the range of from about 0.1 mg/kg to about 10 mg/kg, or in the range of from about 0.5 mg/kg to about 10 mg/kg, or in the range of from about 0.5 mg/kg to about 5 mg/kg, or in the range of from about 0.5 mg/kg to about 3 mg/kg, or in the range of from about 0.5 mg/kg to about 2 mg/kg. In certain embodiments, the anti-CTLA-4 antibody component of the neoadjuvant combination therapy is administered at a dose of less than about 3 mg/kg, or less than about 2 mg/kg. Sometimes, the anti-CTLA-4 component of the neoadjuvant combination therapy is administered at a dose selected from the group consisting of about 1 mg/kg and about 2 mg/kg. Often, the anti-CTLA-4 antibody component of the neoadjuvant combination therapy is administered at a dose of about 1 mg/kg.

In a specific embodiment, the present invention provides a method of treating, alleviating a symptom of, and/or delaying the progression of a cancer in a subject having a solid tumor, the method comprising administering to the subject a neoadjuvant combination therapy comprising

(A) an activatable anti-PDL1 antibody at a fixed dose of 800 mg or about 800 mg, wherein the activatable anti-PDL1 antibody comprises:

(i) an antibody or antigen-binding portion thereof that binds human PDL1 (AB) that comprises:

a heavy chain variable region (VH) comprising a complementarity determining region 1 (CDRH1) that comprises the amino acid sequence of SEQ ID NO:125, a complementarity determining region 2 (CDRH2) that comprises the amino acid sequence of SEQ ID NO:126, and a complementarity determining region 3 (CDRH3) that comprises the amino acid sequence of SEQ ID NO:127, and

a light chain variable region (VL) comprising a light chain complementarity determining region 1 (CDRL1) that comprises the amino acid sequence of SEQ ID NO:128, a light chain complementarity determining region 2 (CDRL2) that comprises the amino acid sequence of SEQ ID NO:129, and a light chain complementarity determining region 3 (CDRL3) that comprises the amino acid sequence of SEQ ID NO:130;

(ii) a cleavable moiety (CM) linked, directly or indirectly, to the AB, wherein the CM is a polypeptide that functions as a substrate for a protease; and

(iii) a masking moiety (MM) linked, directly or indirectly, to the CM; and

(B) an anti-CTLA-4 antibody at a dose of 1 mg/kg.

Suitable MM, CM, spacer, and linker amino acid sequences include any of those described herein. In some of these embodiments, the activatable anti-PDL1 antibody comprises a heavy chain variable region (VH) comprising the amino acid sequence of SEQ ID NO:118, and a light chain variable region (VL) comprising the amino acid sequence of SEQ ID NO:119. In certain of these embodiments, the activatable anti-PDL1 antibody comprises a light chain and a heavy chain, wherein the light chain comprises the MM, the CM, and the VL, and wherein the light chain comprises the amino acid sequence of SEQ ID NO:120, and wherein the heavy chain comprises the VH which comprises the amino acid sequence of SEQ ID NO:118, or comprises a light chain and a heavy chain, and wherein the light chain comprises a spacer, the MM, the CM, and the VL, and wherein the light chain comprises the amino acid sequence of SEQ ID NO:121, and wherein the heavy chain comprises the VH which comprises the amino acid sequence of SEQ ID NO:118.

In another specific embodiment, the present invention provides a method of treating, alleviating a symptom of, and/or delaying the progression of a cancer in a subject having a solid tumor, the method comprising administering to the subject a neoadjuvant combination therapy comprising

(A) an activatable anti-PDL1 antibody at a fixed dose of 800 mg or about 800 mg,

wherein the activatable anti-PDL1 antibody comprises a heavy chain comprising the amino acid sequence of SEQ ID NO:122, and a light chain comprising an amino acid sequence selected from the group consisting of SEQ ID NO:123 and SEQ ID NO:124, wherein the activatable anti-PDL1 antibody comprises an antibody or antigen-binding portion thereof that binds human PDL1 (AB), a cleavable moiety (CM), and a masking moiety (MM); and

(B) an anti-CTLA-4 antibody at a dose of 1 mg/kg. In some of these embodiments, the light chain comprises the amino acid sequence of SEQ ID NO:123. In other embodiments, the light chain comprises the amino acid sequence of SEQ ID NO:124.

In certain embodiments, following completion of both the administration of one or more doses of the neoadjuvant combination therapy and the procedure of surgically resecting all or a portion of the solid tumor, the method further comprises administering to the subject one or more doses of a post-surgical combination therapy comprising a dose of the activatable anti-PDL1 antibody and a dose of the anti-CTLA-4 antibody. The activatable anti-PDL1 antibody and anti-CTLA-4 antibody employed in the neoadjuvant and post-surgical combination therapies are typically the same.

In some embodiments, multiple doses of the post-surgical combination therapy are administered to the subject at a frequency of one dose of the post-surgical combination therapy per interval of time over a first post-surgical period of time. For example, two, three, four, five, six, seven, eight, nine, or ten or more doses of the post-surgical combination therapy may be administered to the subject. Typically, a dose of the post-surgical combination therapy is administered once every three weeks (21 days). In some embodiments, a dose of the post-surgical combination therapy is administered once every week. In some embodiments, a dose of the post-surgical combination therapy is administered once every two weeks. In some embodiments, a dose of the post-surgical combination therapy is administered once every four weeks. In certain of these embodiments, following surgical resection of the tumor, two doses of the post-surgical combination therapy are administered to the subject at a frequency of one dose per interval of time over (a first) post-surgical period of time. In some embodiments, doses of the post-surgical combination therapy are administered at a constant frequency post-surgery (e.g., two or more doses of the post-surgical combination therapy can be administered once every 3 weeks). In some embodiments, doses of the post-surgical combination therapy are administered at a variable frequency post-surgery (e.g., the time period between the first two doses of the post-surgical combination therapy can 3 weeks, and future doses of the post-surgical combination therapy can be administered weekly). As will be understood by a person of ordinary skill in the art, other constant and variable dosing periods for the post-surgical combination therapy described herein can be employed. Often, administration of the first dose of the post-surgical combination therapy is administered one, two, three, four, five, six, seven, eight, nine, ten, eleven, or twelve weeks following surgical resection of the tumor. In certain embodiments, the first dose of the post-surgical combination therapy is administered at a time point in the range of from four to eight weeks or from five to seven weeks following the procedure of surgically resecting all or a portion of the tumor. In some embodiments, the first dose of the post-surgical combination therapy is administered about six weeks following surgical resection of the tumor.

In certain of these embodiments, the activatable anti-PDL1 antibody component of the post-surgical combination therapy is administered at a fixed dose in the range of from 240 mg to 2400 mg. In some embodiments, the activatable anti-PDL1 antibody component of the post-surgical combination therapy is administered at a fixed dose of 800 mg. In other embodiments, the activatable anti-PDL1 antibody component of the post-surgical combination therapy is administered to the subject at a dose in the range of from 0.3 mg/kg to 30 mg/kg. In some embodiments, the activatable anti-PDL1 antibody component of the post-surgical combination therapy is administered to the subject at a dose of 0.3 mg/kg, 1.0 mg/kg, 3.0 mg/kg, 10.0 mg/kg or 30.0 mg/kg. In certain of these embodiments, the activatable anti-PDL1 antibody component of the post-surgical combination therapy is administered at a fixed dose in the range of from about 240 mg to about 2400 mg. In some embodiments, the activatable anti-PDL1 antibody component of the post-surgical combination therapy is administered at a fixed dose of about 800 mg. In other embodiments, the activatable anti-PDL1 antibody component of the post-surgical combination therapy is administered to the subject at a dose in the range of from about 0.3 mg/kg to about 30 mg/kg. In some embodiments, the activatable anti-PDL1 antibody component of the post-surgical combination therapy is administered to the subject at a dose of about 0.3 mg/kg, about 1.0 mg/kg, about 3.0 mg/kg, about 10.0 mg/kg or about 30.0 mg/kg.

In some embodiments, the anti-CTLA-4 antibody component of the post-surgical combination therapy is administered at a dose in the range of from 0.3 mg/kg to 30 mg/kg. Sometimes the anti-CTLA-4 antibody component of the post-surgical combination therapy is administered at a dose in the range of from 0.1 mg/kg to 20 mg/kg, or in the range of from 0.1 mg/kg to 15 mg/kg, or in the range of from 0.1 mg/kg to 10 mg/kg, or in the range of from 0.5 mg/kg to 10 mg/kg, or in the range of from 0.5 mg/kg to 5 mg/kg, or in the range of from 0.5 mg/kg to 3 mg/kg, or in the range of from 0.5 mg/kg to 2 mg/kg. In certain embodiments, the anti-CTLA-4 antibody component of the post-surgical combination therapy is administered at a dose of less than 3 mg/kg, or less than 2 mg/kg. Sometimes, the anti-CTLA-4 antibody component of the post-surgical combination therapy is administered at a dose selected from the group consisting of 1 mg/kg and 2 mg/kg. Often, the anti-CTLA-4 antibody component of the post-surgical combination therapy is administered at a dose of 1 mg/kg. In certain embodiments, the activatable anti-PDL1 antibody component of the post-surgical combination therapy is administered to the subject at a fixed dose of 800 mg and the anti-CTLA-4 antibody component of the post-surgical combination therapy is administered to the subject at a dose of 1 mg/kg. In some embodiments, the anti-CTLA-4 antibody component of the post-surgical combination therapy is administered at a dose in the range of from about 0.3 mg/kg to about 30 mg/kg. Sometimes the anti-CTLA-4 antibody component of the post-surgical combination therapy is administered at a dose in the range of from about 0.1 mg/kg to about 20 mg/kg, or in the range of from about 0.1 mg/kg to about 15 mg/kg, or in the range of from about 0.1 mg/kg to about 10 mg/kg, or in the range of from about 0.5 mg/kg to about 10 mg/kg, or in the range of from about 0.5 mg/kg to about 5 mg/kg, or in the range of from about 0.5 mg/kg to about 3 mg/kg, or in the range of from about 0.5 mg/kg to about 2 mg/kg. In certain embodiments, the anti-CTLA-4 antibody component of the post-surgical combination therapy is administered at a dose of less than about 3 mg/kg, or less than about 2 mg/kg. Sometimes, the anti-CTLA-4 antibody component of the post-surgical combination therapy is administered at a dose selected from the group consisting of about 1 mg/kg and about 2 mg/kg. Often, the anti-CTLA-4 antibody component of the post-surgical combination therapy is administered at a dose of about 1 mg/kg. In certain embodiments, the activatable anti-PDL1 antibody component of the post-surgical combination therapy is administered to the subject at a fixed dose of about 800 mg and the anti-CTLA-4 antibody component of the post-surgical combination therapy is administered to the subject at a dose of about 1 mg/kg.

In some embodiments, a post-surgical regimen of administering activatable anti-PDL1 antibody as a monotherapy is employed. Typically, the activatable anti-PDL1 antibody is the same as that employed in the neoadjuvant and post-surgical combination therapies. In these embodiments, one or more doses of the activatable anti-PDL1 antibody is administered to the subject as a monotherapy following administration of the one or more doses of the post-surgical combination therapy. In some embodiments, the first dose of the post-surgical monotherapy is administered at least 1 week following administration of the last dose of the post-surgical combination therapy. In some embodiments, the first dose of the post-surgical monotherapy is administered at least 2 weeks following administration of the last dose of the post-surgical combination therapy. In some embodiments, the first dose of the post-surgical monotherapy is administered at least 3 weeks following administration of the last dose of the post-surgical combination therapy. In some embodiments, the first dose of the post-surgical monotherapy is administered at least 4 weeks following administration of the last dose of the post-surgical combination therapy. In certain embodiments, the first dose of the post-surgical monotherapy is administered 1 week following administration of the last dose of the post-surgical combination therapy. In certain embodiments, the first dose of the post-surgical monotherapy is administered 2 weeks following administration of the last dose of the post-surgical combination therapy. In certain embodiments, the first dose of the post-surgical monotherapy is administered 3 weeks following administration of the last dose of the post-surgical combination therapy. In certain embodiments, the first dose of the post-surgical monotherapy is administered 4 weeks following administration of the last dose of the post-surgical combination therapy.

Often, multiple doses of the post-surgical monotherapy are administered to the subject at a frequency of one dose of the activatable anti-PDL1 antibody per interval of time over a second post-surgical period of time. For example, two, three, four, five, six, seven, eight, nine, or ten or more doses of the post-surgical monotherapy may be administered to the subject. In some embodiments, multiple doses of the post-surgical monotherapy are administered to the subject at a frequency of one dose of the activatable anti-PDL1 antibody every 2 weeks. Monotherapy treatment may continue until the subject no longer exhibits improvement. In some embodiments, monotherapy treatment continues for up to one year. Typically, multiple doses of the activatable anti-PDL1 antibody as a monotherapy are administered to the subject. In some embodiments, the monotherapy dose is administered once every two weeks. In some embodiments, multiple doses of the activatable anti-PDL1 antibody are administered to the subject as a monotherapy at a constant frequency over the second post-surgical period of time (e.g., two or more doses of the activatable anti-PDL1 antibody are administered to the subject as a monotherapy can be administered once every two weeks). In some embodiments, multiple doses of the activatable anti-PDL1 antibody are administered to the subject as a monotherapy at a variable frequency over the second post-surgical period of time (e.g., the time period between the first two doses of the activatable anti-PDL1 antibody can two weeks, and future doses of the activatable anti-PDL1 antibody can be administered weekly or monthly). As will be understood by a person of ordinary skill in the art, other constant and variable dosing periods for the activatable anti-PDL1 antibody described herein can be employed.

In some embodiments, a post-surgical regimen of administering activatable anti-PDL1 antibody as a monotherapy is employed in the absence of prior administration of a post-surgical combination therapy. Typically, the activatable anti-PDL1 antibody is the same as that employed in the neoadjuvant combination therapy. In these embodiments, one or more doses of the activatable anti-PDL1 antibody is administered to the subject as a monotherapy following surgery. In some embodiments, the first dose of the post-surgical monotherapy is administered at least 1 week following surgery. In some embodiments, the first dose of the post-surgical monotherapy is administered at least 2 weeks following surgery. In some embodiments, the first dose of the post-surgical monotherapy is administered at least 3 weeks following surgery. In some embodiments, the first dose of the post-surgical monotherapy is administered at least 4 weeks following surgery. In certain embodiments, the first dose of the post-surgical monotherapy is administered 1 week following surgery. In certain embodiments, the first dose of the post-surgical monotherapy is administered 2 weeks following surgery. In certain embodiments, the first dose of the post-surgical monotherapy is administered 3 weeks following surgery. In certain embodiments, the first dose of the post-surgical monotherapy is administered 4 weeks following surgery.

Often, multiple doses of the post-surgical monotherapy are administered to the subject at a frequency of one dose of the activatable anti-PDL1 antibody per interval of time over a first post-surgical period of time. For example, two, three, four, five, six, seven, eight, nine, or ten or more doses of the post-surgical monotherapy may be administered to the subject. In some embodiments, multiple doses of the post-surgical monotherapy are administered to the subject at a frequency of one dose of the activatable anti-PDL1 antibody every 2 weeks. Monotherapy treatment may continue until the subject no longer exhibits improvement. In some embodiments, monotherapy treatment continues for up to one year. Typically, multiple doses of the activatable anti-PDL1 antibody as a monotherapy are administered to the subject. In some embodiments, the monotherapy dose is administered once every two weeks. In some embodiments, multiple doses of the activatable anti-PDL1 antibody are administered to the subject as a monotherapy at a constant frequency over the first post-surgical period of time (e.g., two or more doses of the activatable anti-PDL1 antibody are administered to the subject as a monotherapy can be administered once every two weeks). In some embodiments, multiple doses of the activatable anti-PDL1 antibody are administered to the subject as a monotherapy at a variable frequency over the first post-surgical period of time (e.g., the time period between the first two doses of the activatable anti-PDL1 antibody can two weeks, and future doses of the activatable anti-PDL1 antibody can be administered weekly or monthly). As will be understood by a person of ordinary skill in the art, other constant and variable dosing periods for the activatable anti-PDL1 antibody described herein can be employed.

In some embodiments, the dose of activatable anti-PDL1 antibody administered to the subject as a monotherapy (with or without prior administration of a post-surgical combination therapy) is a fixed dose in the range of from 240 mg to 2400 mg. In some embodiments, when administered as a monotherapy, the activatable anti-PDL1 antibody is administered at a fixed dose of 800 mg. In other embodiments, when administered as a monotherapy, the activatable anti-PDL1 antibody is administered to the subject at a dose in the range of from 0.3 mg/kg to 30 mg/kg. In certain of these embodiments, the activatable anti-PDL1 antibody is administered to the subject as a monotherapy at a dose of 0.3 mg/kg, 1.0 mg/kg, 3.0 mg/kg, 10.0 mg/kg or 30.0 mg/kg. Typically, multiple doses of the post-surgical monotherapy, each a fixed dose of 800 mg of the activatable anti-PDL1 antibody, are administered to the subject every 2 weeks. In some embodiments, the dose of activatable anti-PDL1 antibody administered to the subject as a monotherapy (with or without prior administration of a post-surgical combination therapy) is a fixed dose in the range of from about 240 mg to about 2400 mg. In some embodiments, when administered as a monotherapy, the activatable anti-PDL1 antibody is administered at a fixed dose of about 800 mg. In some embodiments, when administered as a monotherapy, the activatable anti-PDL1 antibody is administered to the subject at a dose in the range of from about 0.3 mg/kg to about 30 mg/kg. In certain of these embodiments, the activatable anti-PDL1 antibody is administered to the subject as a monotherapy at a dose of about 0.3 mg/kg, about 1.0 mg/kg, about 3.0 mg/kg, about 10.0 mg/kg or about 30.0 mg/kg. Typically, multiple doses of the post-surgical monotherapy, each a fixed dose of about 800 mg of the activatable anti-PDL1 antibody, are administered to the subject every 2 weeks.

The same protocols with respect to routes of administration, duration of administration, and order of administration may be used when administrating the activatable anti-PDL1 antibody and anti-CTLA-4 components of the neoadjuvant and post-surgical combination therapies. Typically, when administered as a component of a combination therapy (i.e., either a neoadjuvant or post-surgical combination therapy), the activatable anti-PDL1 antibody is administered to the subject prior to administering the anti-CTLA-4 antibody. In certain embodiments, the anti-CTLA-4 antibody is administered to the subject no sooner than 30 minutes after completion of the administration of the activatable anti-PDL1 antibody component of either the neoadjuvant combination therapy or the post-surgical combination therapy. Often, the components of the neoadjuvant combination therapy (i.e., the activatable anti-PDL1 antibody and the anti-CTLA-4 antibody) are administered to the subject on the same day. Likewise, the components of the post-surgical combination therapy (i.e., the activatable anti-PDL1 antibody and the anti-CTLA-4 antibody) are often administered on the same day. In some embodiments, the activatable anti-PDL1 antibody is administered to the subject by intravenous (IV) infusion. Similarly, in some embodiments, the anti-CTLA-4 antibody is administered to the subject by intravenous infusion. Typically, both the activatable anti-PDL1 antibody and the anti-CTLA-4 antibody are administered to the subject intravenously (e.g., by intravenous infusion).

In some embodiments, when administered as a component of a combination therapy (i.e., either a neoadjuvant or post-surgical combination therapy), the activatable anti-PDL1 antibody is administered to the subject after administering the anti-CTLA-4 antibody. In certain embodiments, the activatable anti-PDL1 antibody is administered to the subject no sooner than 30 minutes after completion of the administration of the activatable anti-CTLA-4 antibody component of either the neoadjuvant combination therapy or the post-surgical combination therapy. Often, the components of the neoadjuvant combination therapy (i.e., the activatable anti-PDL1 antibody and the anti-CTLA-4 antibody) are administered to the subject on the same day. Likewise, the components of the post-surgical combination therapy (i.e., the activatable anti-PDL1 antibody and the anti-CTLA-4 antibody) are often administered on the same day. In some embodiments, the activatable anti-PDL1 antibody is administered to the subject by intravenous (IV) infusion. Similarly, in some embodiments, the anti-CTLA-4 antibody is administered to the subject by intravenous infusion. Typically, both the activatable anti-PDL1 antibody and the anti-CTLA-4 antibody are administered to the subject intravenously (e.g., by intravenous infusion).

The same protocols with respect to routes of administration, duration of administration, and order of administration may be used when administrating the activatable anti-PDL1 antibody post-surgical monotherapy. In some embodiments, the activatable anti-PDL1 antibody post-surgical monotherapy is administered to the subject intravenously (e.g., by intravenous infusion).

In a specific embodiment, administering the combination therapy (e.g., either the neoadjuvant or post-surgical combination therapy) comprises:

(i) administering the activatable anti-PDL1 antibody by intravenous infusion over a period of 60 minutes or about 60 minutes;

(ii) administering a saline flush; and

(iii) administering the anti-CTLA-4 antibody by intravenous infusion over a period of 30 minutes or about 30 minutes,

wherein the step of administering the anti-CTLA-4 antibody is carried out no sooner than 30 minutes or about 30 minutes after completion of the step of administering the activatable anti-PDL1 antibody.

In a specific embodiment, administering the combination therapy (e.g., either the neoadjuvant or post-surgical combination therapy) comprises:

(i) administering the anti-CTLA-4 antibody by intravenous infusion over a period of 30 minutes or about 30 minutes,

(ii) administering a saline flush; and

(iii) administering the activatable anti-PDL1 antibody by intravenous infusion over a period of 60 minutes or about 60 minutes;

wherein the step of administering the activatable anti-PDL1 antibody is carried out no sooner than 30 minutes or about 30 minutes after completion of the step of administering the anti-CTLA-4 activatable anti-PDL1 antibody.

In some embodiments, the activatable anti-PDL1 antibody is administered by intravenous infusion over a period of 30, 35, 40, 45, 50, 55, 60, 65, 70, 75, 80, 85, 90, 95, 100, 105, 110, 115, or 120 minutes. In some embodiments, the activatable anti-PDL1 antibody is administered by intravenous infusion over a period of about 30, about 35, about 40, about 45, about 50, about 55, about 60, about 65, about 70, about 75, about 80, about 85, about 90, about 95, about 100, about 105, about 110, about 115, or about 120 minutes. In some embodiments, the anti-CTLA-4 antibody is administered by intravenous infusion over a period of 15, 20, 25, 30, 35, 40, 45, 50, 55, or 60 minutes. In some embodiments, the anti-CTLA-4 antibody is administered by intravenous infusion over a period of about 15, about 20, about 25, about 30, about 35, about 40, about 45, about 50, about 55, or about 60 minutes. In some embodiments, the anti-CTLA-4 antibody is administered no sooner than 15, 20, 25, 30, 35, 40, 45, 50, 55, or 60 minutes after completion of the step of administering the activatable anti-PDL1 antibody. In some embodiments, the anti-CTLA-4 antibody is administered no sooner than about 15, about 20, about 25, about 30, about 35, about 40, about 45, about 50, about 55, or about 60 minutes after completion of the step of administering the activatable anti-PDL1 antibody. In some embodiments, the activatable anti-PDL1 antibody is administered no sooner than 15, 20, 25, 30, 35, 40, 45, 50, 55, or 60 minutes after completion of the step of administering the anti-CTLA-4 antibody. In some embodiments, the activatable anti-PDL1 antibody is administered no sooner than about 15, about 20, about 25, about 30, about 35, about 40, about 45, about 50, about 55, or about 60 minutes after completion of the step of administering the anti-CTLA-4 antibody.

In a specific embodiment, the method comprises administering 2 doses of the neoadjuvant combination therapy to the subject at a frequency of one dose every 3 weeks, and administering two doses of the post-surgical combination therapy to the subject at a frequency of one dose every 3 weeks over a first post-surgical period of time, and administering multiple doses of the post-surgical monotherapy to the subject at a frequency of one dose every 2 weeks over a second post-surgical period of time, wherein the neoadjuvant combination therapy and post-surgical combination therapy each comprise a fixed dose of 800 mg of the activatable anti-FPDL1 antibody and a dose of 1 mg/kg of the anti-CTLA-4 antibody, and wherein the post-surgical monotherapy comprises a fixed dose of 800 mg of the activatable anti-PDL1 antibody. In another specific embodiment, the method comprises administering 2 doses of the neoadjuvant combination therapy to the subject at a frequency of one dose about every 3 weeks, and administering two doses of the post-surgical combination therapy to the subject at a frequency of one dose about every 3 weeks over a first post-surgical period of time, and administering multiple doses of the post-surgical monotherapy to the subject at a frequency of one dose about every 2 weeks over a second post-surgical period of time, wherein the neoadjuvant combination therapy and post-surgical combination therapy each comprise a fixed dose of about 800 mg of the activatable anti-PDL1 antibody and a dose of about 1 mg/kg of the anti-CTLA-4 antibody, and wherein the post-surgical monotherapy comprises a fixed dose of about 800 mg of the activatable anti-PDL1 antibody.

Anti-CTLA-4 antibodies that are suitable for use in the methods and treatments described herein include any anti-CTLA-4 antibody having binding specificity for human CTLA-4. Typically, the anti-CTLA-4 antibody is ipilimumab. Ipilimumab is a fully human, IgG1 monoclonal antibody that blocks the binding of CTLA-4 to its B7 ligands, and is marketed as YERVOY. In some embodiments, the anti-CTLA-4 antibody is tremelimumab (also referred to as ticilimumab or CP-675,206), a fully human IgG2 monoclonal antibody that blocks the binding of CTLA-4 to its B7 ligands (see, e.g., Lee et al., J Gynecol Oncol. 2019 November; 30(6):e112. doi: 10.3802/jgo.2019.30.e112, incorporated herein by reference in its entirety). In some embodiments, the anti-CTLA-4 antibody is CS1002, a fully human IgG1 monoclonal antibody that blocks the binding of CTLA-4 to its B7 ligands. In some embodiments, the anti-CTLA-4 antibody is zalifrelimab (also referred to as AGEN1884) an IgG1 monoclonal antibody. In some embodiments, the anti-CTLA-4 antibody is ADU-1604, a humanized IgG2 monoclonal antibody. In some embodiments, the anti-CTLA-4 antibody is CBT-509, a novel IgG1 humanized monoclonal antibody (see, e.g., Shi et al., DOI: 10.1200/JCO.2019.37.8_suppl.32 Journal of Clinical Oncology 37, no. 8_suppl (Mar. 10, 2019) 32-32, incorporated herein by reference in its entirety). Other anti-CTLA-4 antibodies are contemplated for use with the methods and materials described herein, e.g., any of the anti-CTLA-4 antibodies disclosed in Waight et al. (Cancer Cell. 2018 Jun. 11; 33(6): 1033-1047.e5, doi: 10.1016/j.ccell.2018.05.005, incorporated herein by reference in its entirety), or any anti-CTLA-4 antibody that a person of ordinary skill in the art could find by searching the clinicaltrials.gov website.

Subjects employed in the practice of these methods are typically under the care of a physician, and have typically been diagnosed as having a solid tumor. In some embodiments, the methods comprise a further step of surgically resecting all or a portion of the solid tumor in the subject following administration of the last dose of the neoadjuvant combination therapy. In certain embodiments, following the step of surgically resecting all or a portion of the solid tumor, the method further comprises administering one or more doses of a post-surgical combination therapy in accordance with the methods described herein. In some embodiments, the method further comprises administering one or more doses of a post-surgical monotherapy in accordance with the methods described herein.

In the embodiments described herein, the cancer is typically a melanoma. The melanoma may be resectable Stage III melanoma. The resectable Stage III melanoma may be confirmed by histological or cytological assessment.

An illustrative treatment regimen utilizing the above-described combination therapy is described in Example 1.

The activatable anti-PDL1 antibody and anti-CTLA-4 antibody employed in the methods of the invention can be formulated into pharmaceutical compositions suitable for intravenous administration. Each may be provided in lyophilized or solution form, but if either compound is provided in lyophilized form it is solubilized in a pharmaceutically acceptable diluent prior to administration. For intravenous administration, suitable diluents include physiological saline, bacteriostatic water, Cremophor EL™ (BASF, Parsippany, N.J.), phosphate buffered saline (PBS), and the like. Pharmaceutical compositions comprising activatable anti-PDL1 antibody that are suitable for use in the practice of the present invention are described in PCT Pub. Nos. WO 2016/149201 and WO 2018/222949, each of which is incorporated herein by reference. In all cases, the composition must be sterile.

In a further embodiment, the present invention provides an activatable anti-PDL1 antibody, or composition comprising an activatable anti-PDL1 antibody and a pharmaceutically acceptable diluent, for use in the treatment of a cancer, wherein the treatment comprises administering the activatable anti-PDL1 antibody, or composition thereof, intravenously to a subject in a neoadjuvant combination with an anti-CTLA-4 antibody that is administered intravenously to the subject,

wherein the activatable anti-PDL1 antibody comprises:

(i) an antibody or antigen-binding portion thereof that binds human PDL1 (AB) that comprises:

a heavy chain variable region (VH) comprising a complementarity determining region 1 (CDRH1) that comprises the amino acid sequence of SEQ ID NO:125, a complementarity determining region 2 (CDRH2) that comprises the amino acid sequence of SEQ ID NO:126, and a complementarity determining region 3 (CDRH3) that comprises the amino acid sequence of SEQ ID NO:127, and

a light chain variable region (VL) comprising a light chain complementarity determining region 1 (CDRL1) that comprises the amino acid sequence of SEQ ID NO:128, a light chain complementarity determining region 2 (CDRL2) that comprises the amino acid sequence of SEQ ID NO:129, and a light chain complementarity determining region 3 (CDRL3) that comprises the amino acid sequence of SEQ ID NO:130;

(ii) a cleavable moiety (CM) linked, directly or indirectly, to the AB, wherein the CM is a polypeptide that functions as a substrate for a protease; and

(iii) a masking moiety (MM) linked, directly or indirectly, to the CM; and

(B) an anti-CTLA-4 antibody,

wherein the subject has a solid tumor.

Amino acid sequences encoding CM, MM, VL, VH, linker, and spacer components that are suitable for use in the structure of the above-described activatable anti-PDL1 antibody include any of those described hereinabove.

In a further embodiment, the present invention provides an activatable anti-PDL1 antibody for use in the treatment of a cancer, wherein the treatment comprises administering the activatable anti-PDL1 antibody intravenously to a subject at a fixed dose of 800 mg in a neoadjuvant combination with an anti-CTLA-4 antibody that is administered intravenously to the subject at a dose of 1 mg/kg,

wherein the activatable anti-PDL1 antibody comprises a heavy chain comprising the amino acid sequence of SEQ ID NO:122, and a light chain comprising the amino acid sequence selected from the group consisting of SEQ ID NO:123 and SEQ ID NO:124,

wherein the activatable anti-PDL1 antibody comprises an antibody or antigen-binding portion thereof that binds human PDL1 (AB), a cleavable moiety (CM), and a masking moiety (MM), and

wherein the subject has a solid tumor. In some embodiments, the light chain comprises the amino acid sequence of SEQ ID NO:123. In other embodiments, the light chain comprises the amino acid sequence of SEQ ID NO:124.

In some embodiments, provided herein is an activatable anti-PDL1 antibody, or composition comprising an activatable anti-PDL1 antibody and a pharmaceutically acceptable diluent, for use in the treatment of a cancer, wherein the treatment comprises administering the activatable anti-PDL1 antibody, or composition thereof, intravenously to a subject in a post-surgical combination with an anti-CTLA-4 antibody that is administered intravenously to the subject. In some embodiments, the activatable anti-PDL1 antibody and anti-CTLA-4 antibody employed in the neoadjuvant and post-surgical combination therapies are the same.

In some embodiments, provided herein is an activatable anti-PDL1 antibody, or composition comprising an activatable anti-PDL1 antibody and a pharmaceutically acceptable diluent, for use in the treatment of a cancer, wherein the treatment comprises administering the activatable anti-PDL1 antibody, or composition thereof, intravenously to a subject as a post-surgical monotherapy after administration of the neoadjuvant and post-surgical combination therapies. In some embodiments, the activatable anti-PDL1 antibody employed in the neoadjuvant combination therapy, the post-surgical combination therapy, and the post-surgical monotherapy are the same.

Doses and/or dosing regimens of each of the activatable anti-PDL1 antibody and the anti-CTLA-4 antibody components of the neoadjuvant combination, the activatable anti-PDL1 antibody and the anti-CTLA-4 antibody components of the post-surgery combination therapy, or the activatable anti-PDL1 antibody of the post-surgery monotherapy that are suitable for use in the above-described treatment include any of those described herein for the corresponding methods of treatment. In some embodiments, the cancer is a melanoma, such as, for example, resectable Stage III melanoma. Typically, the anti-CTLA-4 antibody is ipilimumab.

Activatable anti-PDL1 antibodies for use in the treatment of cancer may comprise any of the treatment steps described herein.

The following example further illustrates the practice of the invention, but should not be construed as limiting its scope in any way.

### EXAMPLES

### Evaluation of an Activatable Anti-PDL1 Antibody in Combination with an Anti-CTLA-4 Antibody as a Neoadjuvant Combination Therapy for Subjects with Solid Tumor

This study evaluates the antitumor effect of PL07-2001-C5H9v2 in combination with ipilimumab in subjects with solid tumors based on pathologic response following neoadjuvant administration of the neoadjuvant combination therapy.

PL07-2001-C5H9v2 is a protease activatable anti-PDL1 antibody that comprises the heavy chain sequence of SEQ ID NO:122 and the light chain sequence of SEQ ID NO:124. PL07-2001-C5H9v2 comprises two heavy chains and two light chains. The light chain contains a prodomain sequence that comprises a MM and a CM. See WO 2016/149201 and WO 2018/222949. The corresponding activated anti-PDL1 antibody binds human PDL1.

Ipilimumab is an anti-CTLA-4 antibody. It is a fully human, IgG1 monoclonal antibody that blocks the binding of CTLA-4 to its B7 ligands and is marketed as YERVOY.

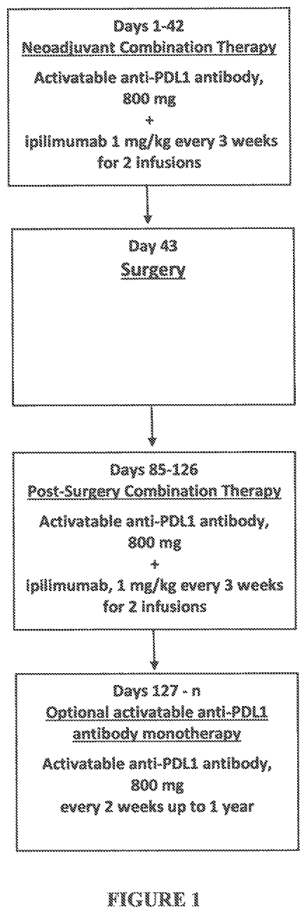

In this study, subjects will be treated with 2 doses of 800 mg PL07-2001-C5H9v2 plus 1 mg/kg ipilimumab combination (i.e., q3w on Day 1 and Day 22; all ±2 days), followed by surgical resection of the tumor on Day 43 (−2/+7 days). An additional 2 doses of 800 mg P07-2001-C5H9v2 plus 1 mg/kg ipilimumab combination will be administered approximately 6 weeks post-surgery (i.e., q3w on Day 85 (±2 days) and Day 106 (±2 days). Three weeks following receipt of the fourth dose of combination treatment (i.e., Day 127 (±2 days), subjects will have the option to continue with 800 mg PL07-2001-C5H9v2 monotherapy q2w. Subjects may receive up to one year of PL07-2001-C5Hv2 post-surgery (including 2 post surgery combination doses and then as monotherapy) until the occurrence of disease relapse, unacceptable toxicity, or other reason for treatment discontinuation. A maximum of 4 doses of ipilimumab are administered to any subject. A schematic representation of the study design is depicted in FIG. 1.

The 800 mg of activatable anti-PDL1 antibody PL07-2001-C5H9v2 is to be infused over 60 minutes. When administered as a component of the combination therapy, the activatable anti-PDL1 antibody is to be administered first, followed by a saline flush, and then followed by the ipilimumab infusion. Ipilimumab is to be infused no sooner than 30 minutes after completion of the PL07-2001-C5H9v2 (activatable anti-PDL1 antibody) infusion. The 1 mg/kg of ipilimumab is to be administered as a 30 minute IV infusion. A minimum of 14 days is required between infusions of PL07-2001-C5H9v2 and a minimum of 21 days between infusions of ipilimumab. In exceptional circumstances, an infusion may be delayed for up to 7 days.

This study comprises one cohort of subjects: Subjects with histologically confirmed resectable Stage III melanoma with palpable disease suitable for curative surgery

The criteria for subject eligibility are as follows

Sex: Al

Accepts Healthy Volunteers: No

### Inclusion Criteria:

1. At least 18 years of age

2. Measurable disease as defined by RECIST v1.1

3. Eastern Cooperative Oncology Group (ECOG) performance status of ≤1 assessment.

4. Agree to provide tumor tissue and blood samples for biomarker assessment

5. Subjects with treated brain metastases are eligible if the brain metastases are stable (no magnetic resonance imaging (MRI) evidence of progression for at least 8 weeks after treatment is complete and within 28 days prior to first dose of study treatment) and the subject does not require radiation therapy or steroids. Active screening for brain metastases (e.g., brain computed tomography or MRI) is not required.

6. Screening laboratory values must meet all of the following criteria:
i. White blood cells >2000/μL or 2.0×109/L
ii. Neutrophils ≥1500/μL or 1.5×109/L
iii. Platelets ≥100×103/μL or 100×109/L
iv. Hemoglobin ≥9.0 g/dL (may have been transfused) or 90.0 g/L
v. Creatinine ≤2 mg/dL or 176.9 μmol/L OR measured or calculated creatinine clearance (glomerular filtration rate can also be used in place of creatinine or creatinine clearance) >50 mL/min
vi. AST and ALT ≤2.5× upper limit of normal (ULN)
vii. Total bilirubin within ULN (unless diagnosed with Gilbert's syndrome, those subjects must have a total bilirubin ≤3.0 mg/dL or 51.3 μmol/L)
viii. Amylase and lipase ≥1.5×ULN
ix. International normalized ratio (INR) and activated partial thromboplastin time (aPTT)≤1.5×ULN
x. Serum albumin ≥2.5 g/dL

7. Histologically or cytologically confirmed resectable Stage III melanoma with 1 or more macroscopic lymph node metastases (measurable according to RECIST v1.1) that can be biopsied and no history of in-transit metastases within the last 6 months.

8. Lactate dehydrogenase (LDH) within normal range.

### Exclusion Criteria:

1. Treatment with cytotoxic chemotherapy, biologic agents, radiation, immunotherapy, or any investigational agent within 28 days prior to the first dose of study treatment. This interval can be reduced to 2 weeks for subjects who received bone-only radiation therapy or for subjects whose most recent prior therapy was an approved single-agent, small-molecule kinase inhibitor having a half-life of 3 days or less.

2. Prior therapy with a chimeric antigen receptor T cell-containing regimen.

3. History of active autoimmune disease(s) including but not limited to inflammatory bowel diseases, rheumatoid arthritis, autoimmune thyroiditis, autoimmune hepatitis, systemic sclerosis, systemic lupus erythematosus, autoimmune vasculitis, autoimmune neuropathies, type 1 insulin-dependent diabetes mellitus.

4. History of myocarditis regardless of the cause.

5. History of intolerance to prior checkpoint inhibitor therapy defined as the need to discontinue treatment due to an irAE.

6. History of toxic epidermal necrolysis or Stevens-Johnson syndrome.

7. History of any syndrome or medical condition that required treatment with systemic steroids (≥10 mg daily prednisone equivalents) or immunosuppressive medications.

Inhaled or topical steroids are permitted.

8. Baseline corrected QT interval (Qtc) >470 ms.

9. Unresolved acute toxicity CTCAE v5.0 Grade ≥1 (or baseline, whichever is greater) from prior anticancer therapy. Alopecia and other nonacute toxicities are acceptable.

10. History of severe allergic or anaphylactic reactions to human mAb therapy or known hypersensitivity to any activatable antibody therapeutic.

11. Subjects with known human immunodeficiency virus, acquired immune deficiency syndrome, or any related illness.

12. Subjects with acute or chronic hepatitis B or C.

13. History of allogeneic tissue/solid organ transplant, stem cell transplant, or bone marrow transplant.

14. Major surgery (e.g., that required general anesthesia) within 4 weeks prior to the first dose of study treatment or minor surgery (e.g., not involving chest, abdomen, or intracranial structures) or gamma knife treatment (with adequate healing) within 14 days prior to first dose of study treatment (excluding biopsies conducted with local/topical anesthesia) if complete healing is confirmed.

15. History of active malignancy not related to the cancer being treated within the previous 2 years, with the exception of localized cancers that are considered cured and, in the opinion of the investigator, present a low risk for recurrence. These exceptions include, but are not limited to, basal or squamous cell skin cancer, superficial bladder cancer, and carcinoma in situ of the prostate, cervix, or breast.

16. Received a live vaccine within 30 days prior to the first dose of study treatment (e.g., measles, mumps, rubella, chicken pox/zoster, yellow fever, rabies, Bacillus Calmette-Guerin, and typhoid vaccine).

17. Intercurrent illness including, but not limited to ongoing severe aortic stenosis; myocardial infarction or stroke within 24 weeks prior to first dose of study treatment; any of the following within 12 weeks prior to first dose of study treatment: symptomatic congestive heart failure (i.e., New York Heart Association Class III or IV), unstable angina pectoris, or clinically significant and uncontrolled cardiac arrhythmia; nonhealing wound or ulcer within 4 weeks prior to Day 1; and active infection requiring systemic antiviral, antibiotic, or antifungal therapy within 5 days prior to first dose of study treatment.

18. Pleural or pericardial effusion or ascites requiring drainage ≥1 time(s) per month.

19. History of multiple myeloma.

20. Women who are pregnant or breastfeeding.

21. Participating in an ongoing interventional clinical study (e.g., medication, radiation, procedures) unless the subject is only being followed for long-term outcomes.

22. Prior systemic treatment for melanoma.

23. Diagnosis of uveal, ocular, or mucocutaneous melanoma.

The primary criterion for defining evidence of anticancer activity is pathologic response following neoadjuvant therapy based on central review of tumor sample from surgical resection. The criteria for management of subject care and treatment discontinuation are radiographic response assessment (prior to surgery), local pathologic assessment of surgical sample after surgery, or disease relapse. Tumor response as defined by RECIST v1.1 will be assessed prior to surgical resection.

The sequence listing is shown in Table 1 below.

TABLE 1
Sequence Listing

| SEQ ID | | |
|---|---|---|
| NO | DESCRIPTION | SEQUENCE |
| 1 | MM | YCEVSELFVLPWCMG |
| 2 | MM | SCLMHPHYAHDYCYV |
| 3 | MM | LCEVLMLLQHPWCMG |
| 4 | MM | IACRHFMEQLPFCHH |
| 5 | MM | FGPRCGEASTCVPYE |
| 6 | MM | ILYCDSWGAGCLTRP |
| 7 | MM | GIALCPSHFCQLPQT |
| 8 | MM | DGPRCFVSGECSPIG |
| 9 | MM | LCYKLDYDDRSYCHI |
| 10 | MM | PCHPHPYDARPYCNV |
| 11 | MM | PCYWHPFFAYRYCNT |
| 12 | MM | VCYYMDWLGRNWCSS |
| 13 | MM | LCDLFKLREFPYCMG |
| 14 | MM | YLPCHFVPIGACNNK |
| 15 | MM | IFCHMGVVVPQCANY |
| 16 | MM | ACHPHPYDARPYCNV |
| 17 | MM | PCHPAPYDARPYCNV |
| 18 | MM | PCHPHAYDARPYCNV |
| 19 | MM | PCHPHPADARPYCNV |
| 20 | MM | PCHPHPYAARPYCNV |
| 21 | MM | PCHPHPYDAAPYCNV |
| 22 | MM | PCHPHPYDARPACNV |
| 23 | MM | PCHPHPYDARPYCAV |
| 24 | MM | PCHAHPYDARPYCNV |
| 25 | MM | PCHPHPYDARAYCNV |
| 26 | CM | LSGRSDNH |
| 27 | CM | TGRGPSWV |
| 28 | CM | PLTGRSGG |
| 29 | CM | TARGPSFK |
| 30 | CM | NTLSGRSENHSG |
| 31 | CM | NTLSGRSGNHGS |
| 32 | CM | TSTSGRSANPRG |
| 33 | CM | TSGRSANP |
| 34 | CM | VHMPLGFLGP |
| 35 | CM | AVGLLAPP |
| 36 | CM | AQNLLGMV |
| 37 | CM | QNQALRMA |
| 38 | CM | LAAPLGLL |
| 39 | CM | STFPFGMF |
| 40 | CM | ISSGLLSS |
| 41 | CM | PAGLWLDP |
| 42 | CM | VAGRSMRP |
| 43 | CM | VVPEGRRS |
| 44 | CM | ILPRSPAF |
| 45 | CM | MVLGRSLL |
| 46 | CM | QGRAITFI |
| 47 | CM | SPRSIMLA |
| 48 | CM | SMLRSMPL |
| 49 | CM | ISSGLLSGRSDNH |
| 50 | CM | AVGLLAPPGGLSGRSDNH |
| 51 | CM | ISSGLLSSGGSGGSLSGRSDNH |
| 52 | CM | LSGRSGNH |
| 53 | CM | SGRSANPRG |
| 54 | CM | LSGRSDDH |
| 55 | CM | LSGRSDIH |
| 56 | CM | LSGRSDQH |
| 57 | CM | LSGRSDTH |
| 58 | CM | LSGRSDYH |
| 59 | CM | LSGRSDNP |
| 60 | CM | LSGRSANP |
| 61 | CM | LSGRSANI |
| 62 | CM | LSGRSDNI |
| 63 | CM | MIAPVAYR |
| 64 | CM | RPSPMWAY |
| 65 | CM | WATPRPMR |
| 66 | CM | FRLLDWQW |
| 67 | CM | ISSGL |
| 68 | CM | ISSGLLS |
| 69 | CM | ISSGLL |
| 70 | CM | ISSGLLSGRSANPRG |
| 71 | CM | AVGLLAPPTSGRSANPRG |
| 72 | CM | AVGLLAPPSGRSANPRG |
| 73 | CM | ISSGLLSGRSDDH |
| 74 | CM | ISSGLLSGRSDIH |
| 75 | CM | ISSGLLSGRSDQH |
| 76 | CM | ISSGLLSGRSDTH |
| 77 | CM | ISSGLLSGRSDYH |
| 78 | CM | ISSGLLSGRSDNP |
| 79 | CM | ISSGLLSGRSANP |
| 80 | CM | ISSGLLSGRSANI |
| 81 | CM | AVGLLAPPGGLSGRSDDH |
| 82 | CM | AVGLLAPPGGLSGRSDIH |
| 83 | CM | AVGLLAPPGGLSGRSDQH |
| 84 | CM | AVGLLAPPGGLSGRSDTH |
| 85 | CM | AVGLLAPPGGLSGRSDYH |
| 86 | CM | AVGLLAPPGGLSGRSDNP |
| 87 | CM | AVGLLAPPGGLSGRSANP |
| 88 | CM | AVGLLAPPGGLSGRSANI |
| 89 | CM | ISSGLLSGRSDNI |
| 90 | CM | AVGLLAPPGGLSGRSDNI |
| 91 | CM | GLSGRSDNHGGAVGLLAPP |
| 92 | CM | GLSGRSDNHGGVHMPLGFLGP |
| 93 | Linker | GSGGS |
| 94 | Linker | GGGS |
| 95 | Linker | GGSG |
| 96 | Linker | GGSGG |
| 97 | Linker | GSGSG |
| 98 | Linker | GSGGG |
| 99 | Linker | GSSGGSGGSGG |
| 100 | Linker | GSSGGSGGSGGS |
| 101 | Linker | GSSGGSGGSGGSGGGS |
| 102 | Linker | GSSGGSGGSG |
| 103 | Linker | GSSGGSGGSGS |
| 104 | Linker | GGGS |
| 105 | Linker | GSSGT |
| 106 | Linker | GSSG |
| 107 | Linker | GGGSSGGSGGSGG |
| 108 | spacer | QGQSGS |
| 109 | spacer | GQSGS |
| 110 | spacer | QSGS |
| 111 | spacer | QGQSGQG |
| 112 | spacer | GQSGQG |
| 113 | spacer | QSGQG |
| 114 | spacer | SGQG |
| 115 | spacer | QGQSGQ |
| 116 | spacer | GQSGQ |
| 117 | spacer | QSGQ |
| 118 | Anti-PDL1 | EVQLLESGGGLVQPGGSLRLSCAASGFTFSSYAMSWVRQA |
| | Heavy Chain | PGKGLEWVSSIWRNGIVTVYADSVKGRFTISRDNSKNTLY |
| | Variable | LQMNSLRAEDTAVYYCAKWSAAFDYWGQGTLVTVSS |
| | Sequence | |
| 119 | Anti-PDL1 | DIQMTQSPSSLSASVGDRVTITCRASQSISSYLNWYQQKP |
| | Light Chain | GKAPKLLIYAASSLQSGVPSRFSGSGSGTDFTLTISSLQP |
| | Variable | EDFATYYCQQDNGYPSTFGGGTKVEIKR |
| | Sequence | |
| 120 | LC | GIALCPSHFCQLPQTGGGSSGGSGGSGGISSGLLSGRSDN |
| | | HGGSDIQMTQSPSSLSASVGDRVTITCRASQSISSYLNWY |
| | | QQKPGKAPKLLIYAASSLQSGVPSRFSGSGSGTDFTLTIS |
| | | SLQPEDFATYYCQQDNGYPSTFGGGTKVEIKR |
| 121 | LC | QGQSGSGIALCPSHFCQLPQTGGGSSGGSGGSGGISSGLL |
| | | SGRSDNHGGSDIQMTQSPSSLSASVGDRVTITCRASQSIS |
| | | SYLNWYQQKPGKAPKLLIYAASSLQSGVPSRFSGSGSGTD |
| | | FTLTISSLQPEDFATYYCQQDNGYPSTFGGGTKVEIKR |
| 122 | Heavy Chain | EVQLLESGGGLVQPGGSLRLSCAASGFTFSSYAMSWVRQA |
| | Sequence | PGKGLEWVSSIWRNGIVTVYADSVKGRFTISRDNSKNTLY |
| | including VH | LQMNSLRAEDTAVYYCAKWSAAFDYWGQGTLVTVSSASTK |
| | and IgG4 | GPSVFPLAPCSRSTSESTAALGCLVKDYFPEPVTVSWNSG |
| | S228P | ALTSGVHTFPAVLQSSGLYSLSSVVTVPSSSLGTKTYTCN |
| | | VDHKPSNTKVDKRVESKYGPPCPTCPAPEFLGGPSVFLFP |
| | | PKPKDTLMISRTPEVTCVVVDVSQEDPEVQFNWYVDGVEV |
| | | HNAKTKPREEQFNSTYRVVSVLTVLHQDWLNGKEYKCKVS |
| | | NKGLPSSIEKTISKAKGQPREPQVYTLPPSQEEMTKNQVS |
| | | LTCLVKGFYPSDIAVEWESNGQPENNYKTTPPVLDSDGSF |
| | | FLYSRLTVDKSRWQEGNVFSCSVMHEALHNHYTQKSLSLS |
| | | LG |
| 123 | Light chain | GIALCPSHFCQLPQTGGGSSGGSGGSGGISSGLLSGRSDN |
| | sequence | HGGSDIQMTQSPSSLSASVGDRVTITCRASQSISSYLNWY |
| | including | QQKPGKAPKLLIYAASSLQSGVPSRFSGSGSGTDFTLTIS |
| | human kappa | SLQPEDFATYYCQQDNGYPSTFGGGTKVEIKRTVAAPSVF |
| | constant | IFPPSDEQLKSGTASVVCLLNNFYPREAKVQWKVDNALQS |
| | region | GNSQESVTEQDSKDSTYSLSSTLTLSKADYEKHKVYACEV |
| | | THQGLSSPVTKSFNRGEC |
| 124 | Full length | QGQSGSGIALCPSHFCQLPQTGGGSSGGSGGSGGISSGLL |
| | light chain | SGRSDNHGGSDIQMTQSPSSLSASVGDRVTITCRASQSIS |
| | including | SYLNWYQQKPGKAPKLLIYAASSLQSGVPSRFSGSGSGTD |
| | human kappa | FTLTISSLQPEDFATYYCQQDNGYPSTFGGGTKVEIKRTV |
| | constant | AAPSVFIFPPSDEQLKSGTASVVCLLNNFYPREAKVQWKV |
| | domain and | DNALQSGNSQESVTEQDSKDSTYSLSSTLTLSKADYEKHK |
| | spacer | VYACEVTHQGLSSPVTKSFNRGEC |
| 125 | CDRH1 | SYAMS |
| 126 | CDRH2 | SSIWRNGIVTVYADS |
| 127 | CDRH3 | WSAAFDY |
| 128 | CDRL1 | RASQSISSYLN |
| 129 | CDRL2 | AASSLQS |
| 130 | CDRL3 | DNGYPST |

While the foregoing invention has been described in some detail for purposes of clarity and understanding, it will be clear to one skilled in the art from a reading of this disclosure that various changes in form and detail can be made without departing from the true scope of the invention. All publications, patent applications, patents, and other references mentioned herein are incorporated by reference in their entirety. In case of conflict, the present specification, including definitions, will control. It is understood that the materials, examples, and embodiments described herein are for illustrative purposes only and not intended to be limiting and that various modifications or changes in light thereof will be suggested to persons skilled in the art and are to be included within the spirit and scope of the appended claims.

We claim:

1. A method of treating, alleviating a symptom of, or delaying the progression of a cancer in a subject having a solid tumor, the method comprising:
administering to the subject a neoadjuvant combination therapy comprising
(A) an activatable anti-PDL1 antibody, wherein the activatable anti-PDL1 antibody comprises:
(i) an antibody or antigen-binding portion thereof that binds human PDL1 (AB) that comprises:
a heavy chain variable region (VH) comprising a complementarity determining region 1 (CDRH1) that comprises the amino acid sequence of SEQ ID NO:125, a complementarity determining region 2 (CDRH2) that comprises the amino acid sequence of SEQ ID NO:126, and a complementarity determining region 3 (CDRH3) that comprises the amino acid sequence of SEQ ID NO:127, and
a light chain variable region (VL) comprising a light chain complementarity determining region 1 (CDRL1) that comprises the amino acid sequence of SEQ ID NO:128, a light chain complementarity determining region 2 (CDRL2) that comprises the amino acid sequence of SEQ ID NO:129, and a light chain complementarity determining region 3 (CDRL3) that comprises the amino acid sequence of SEQ ID NO:130;
(ii) a cleavable moiety (CM) linked, directly or indirectly, to the AB, wherein the CM is a polypeptide that functions as a substrate for a protease; and
(iii) a masking moiety (MM) linked, directly or indirectly, to the CM; and
(B) an anti-CTLA-4 antibody.

2. The method of claim 1, wherein the activatable anti-PDL1 antibody component of the neoadjuvant combination therapy is administered to the subject at a fixed dose in the range of from 240 mg to 2400 mg.

3. The method of any of claims 1-2, wherein the activatable anti-PDL1 antibody component of the neoadjuvant combination therapy is administered to the subject at a dose in the range of from 0.3 mg/kg to 30 mg/kg.

4. The method of any of claims 1-3, wherein the anti-CTLA-4 antibody component of the neoadjuvant combination therapy is administered to the subject at a dose in the range of from 0.1 mg/kg to 20 mg/kg.

5. The method of any of claims 1-4, wherein the anti-CTLA-4 antibody component of the neoadjuvant combination therapy is administered to the subject at a dose of less than 3 mg/kg.

6. The method of claim 5, wherein the anti-CTLA-4 antibody component of the neoadjuvant combination therapy is administered to the subject at a dose selected from the group consisting of 1 mg/kg and 2 mg/kg.

7. A method of treating, alleviating a symptom of, or delaying the progression of a cancer in a subject having a solid tumor, the method comprising administering to the subject a neoadjuvant combination therapy comprising
(A) an activatable anti-PDL1 antibody at a fixed dose of 800 mg, wherein the activatable anti-PDL1 antibody comprises:
(i) an antibody or antigen-binding portion thereof that binds human PDL1 (AB) that comprises:
a heavy chain variable region (VH) comprising a complementarity determining region 1 (CDRH1) that comprises the amino acid sequence of SEQ ID NO:125, a complementarity determining region 2 (CDRH2) that comprises the amino acid sequence of SEQ ID NO:126, and a complementarity determining region 3 (CDRH3) that comprises the amino acid sequence of SEQ ID NO:127, and
a light chain variable region (VL) comprising a light chain complementarity determining region 1 (CDRL1) that comprises the amino acid sequence of SEQ ID NO:128, a light chain complementarity determining region 2 (CDRL2) that comprises the amino acid sequence of SEQ ID NO:129, and a light chain complementarity determining region 3 (CDRL3) that comprises the amino acid sequence of SEQ ID NO:130;
(ii) a cleavable moiety (CM) linked, directly or indirectly, to the AB, wherein the CM is a polypeptide that functions as a substrate for a protease; and
(iii) a masking moiety (MM) linked, directly or indirectly, to the CM; and
(B) an anti-CTLA-4 antibody at a dose of 1 mg/kg.

8. The method of any of claims 1-7, wherein the MM comprises an amino acid sequence selected from the group consisting of SEQ ID NOs:1-25.

9. The method of any of claims 1-8, wherein the MM comprises the amino acid sequence of SEQ ID NO:7.

10. The method of any of claims 1-9, wherein the CM comprises an amino acid sequence selected from the group consisting of SEQ ID NOs:26-92.

11. The method of any of claims 1-10, wherein the CM comprises the amino acid sequence of SEQ ID NO:49.

12. The method of any of claims 1-11, wherein the activatable anti-PDL1 antibody comprises a heavy chain variable region (VH) comprising the amino acid sequence of SEQ ID NO:118, and a light chain variable region (VL) comprising the amino acid sequence of SEQ ID NO:119.

13. The method of any of claims 1-11, wherein the activatable anti-PDL1 antibody comprises a light chain and a heavy chain, wherein the light chain comprises the MM, the CM, and the VL, and wherein the light chain comprises the amino acid sequence of SEQ ID NO:120, and wherein the heavy chain comprises the VH which comprises the amino acid sequence of SEQ ID NO:118.

14. The method of any of claims 1-11, wherein the activatable anti-PDL1 antibody comprises a light chain and a heavy chain, and wherein the light chain comprises a spacer, the MM, the CM, and the VL, and wherein the light chain comprises the amino acid sequence of SEQ ID NO:121, and wherein the heavy chain comprises the VH which comprises the amino acid sequence of SEQ ID NO:118.

15. A method of treating, alleviating a symptom of, or delaying the progression of a cancer in a subject having a solid tumor, the method comprising administering to the subject a neoadjuvant combination therapy comprising
(A) an activatable anti-PDL1 antibody at a fixed dose of 800 mg,
wherein the activatable anti-PDL1 antibody comprises a heavy chain comprising the amino acid sequence of SEQ ID NO:122, and a light chain comprising an amino acid sequence selected from the group consisting of SEQ ID NO:123 and SEQ ID NO:124,
wherein the activatable anti-PDL1 antibody comprises an antibody or antigen-binding portion thereof that binds human PDL1 (AB), a cleavable moiety (CM), and a masking moiety (MM); and
(B) an anti-CTLA-4 antibody at a dose of 1 mg/kg.

16. The method of claim 15, wherein the light chain comprises the amino acid sequence of SEQ ID NO:123.

17. The method of claim 15, wherein the light chain comprises the amino acid sequence of SEQ ID NO:124.

18. The method of any of claims 1-17, wherein the method comprises administering multiple doses of the neoadjuvant combination therapy to the subject at a frequency of one dose of the neoadjuvant combination therapy per interval of time.

19. The method of claim 18, wherein 2 doses of the neoadjuvant combination therapy are administered to the subject at a frequency of one dose per interval of time.

20. The method of any of claims 18-19, wherein the multiple doses of the neoadjuvant combination therapy are administered to the subject at a frequency of one dose of the neoadjuvant combination therapy every 3 weeks.

21. The method of any of claims 18-20, wherein each dose of the activatable anti-PDL1 antibody in the multiple doses is the same, and each dose of the anti-CTLA-4 antibody in the multiple doses is the same.

22. The method of any of claims 1-21, wherein the method further comprises surgically resecting all or a portion of the solid tumor after administration of a last dose of the neoadjuvant combination therapy.

23. The method of claim 22, wherein the step of surgically resecting all or a portion of the solid tumor occurs 6 weeks after administration of a last dose of the neoadjuvant combination therapy.

24. The method of any of claims 1-23, wherein the activatable anti-PDL1 antibody and the anti-CTLA-4 antibody components of the neoadjuvant combination therapy are both administered by intravenous infusion.

25. The method of any of claims 1-24, wherein administering the neoadjuvant combination therapy to the subject comprises administering the activatable anti-PDL1 antibody component of the neoadjuvant combination therapy prior to administering the anti-CTLA-4 antibody component of the neoadjuvant combination therapy.

26. The method of any of claims 1-25, wherein administering the neoadjuvant combination therapy comprises administering the activatable anti-PDL1 antibody and the anti-CTLA-4 antibody components of the neoadjuvant combination therapy to the subject on the same day.

27. The method of any of claims 1-26, wherein administering the neoadjuvant combination therapy comprises administering the activatable anti-PDL1 antibody component of the neoadjuvant combination therapy by intravenous infusion over a period of 60 minutes.

28. The method of any of claims 1-27, wherein administering the neoadjuvant combination therapy comprises administering the anti-CTLA-4 antibody component of the neoadjuvant combination therapy by intravenous infusion over a period of 30 minutes.

29. The method of any of claims 1-28, wherein administering the neoadjuvant combination therapy comprises administering the anti-CTLA-4 antibody component of the neoadjuvant combination therapy no sooner than 30 minutes after administering the activatable anti-PDL1 antibody component of the neoadjuvant combination therapy.

30. The method of any of claims 1-26, wherein administering the neoadjuvant combination therapy comprises:
(i) administering the activatable anti-PDL1 antibody by intravenous infusion over a period of 60 minutes;
(ii) administering a saline flush; and
(iii) administering the anti-CTLA-4 antibody by intravenous infusion over a period of 30 minutes,
wherein the step of administering the anti-CTLA-4 antibody is carried out no sooner than 30 minutes after completion of the step of administering the activatable anti-PDL1 antibody.

31. The method of any of claims 1-30, wherein the method further comprises administering to the subject one or more doses of a post-surgical combination therapy comprising a dose of the activatable anti-PDL1 antibody and a dose of the anti-CTLA-4 antibody.

32. The method of claim 31, wherein multiple doses of the post-surgical combination therapy are administered to the subject at a frequency of one dose of the post-surgical combination therapy per interval of time over a first post-surgical period of time.

33. The method of any of claims 31-32, wherein two doses of the post-surgical combination therapy are administered to the subject at a frequency of one dose per interval of time over the first post-surgical period of time.

34. The method of any of claims 31-33, wherein multiple doses of the post-surgical combination therapy are administered to the subject at a frequency of one dose of the post-surgical combination therapy every 3 weeks.

35. The method of any of claims 31-34, wherein the activatable anti-PDL1 antibody component of the post-surgical combination therapy is administered to the subject at a fixed dose in the range of from 240 mg to 2400 mg.

36. The method of any of claims 31-34, wherein the activatable anti-PDL1 antibody component of the post-surgical combination therapy is administered to the subject at a dose in the range of from 0.3 mg/kg to 30 mg/kg.

37. The method of any of claims 31-36, wherein the anti-CTLA-4 antibody component of the post-surgical combination therapy is administered to the subject at a dose in the range of from 0.1 mg/kg to 20 mg/kg.

38. The method of any of claims 31-36, wherein the anti-CTLA-4 antibody component of the post-surgical combination therapy is administered to the subject at a dose of less than 3 mg/kg.

39. The method of any of claims 31-38, wherein the anti-CTLA-4 antibody component of the post-surgical combination therapy is administered to the subject at a dose selected from the group consisting of 1 mg/kg and 2 mg/kg.

40. The method of any of claims 31-34, wherein the activatable anti-PDL1 antibody component of the post-surgical combination therapy is administered to the subject at a fixed dose of 800 mg, and the anti-CTLA-4 antibody component of the post-surgical combination therapy is administered to the subject at a dose of 1 mg/kg.

41. The method of any of claims 31-40, wherein a first dose of the post-surgical combination therapy is administered to the subject within a time period in the range of from 4 to 8 weeks or in the range of from 5 to 7 weeks following the step of surgically resecting all or a portion of the tumor.

42. The method of any of claims 31-41, wherein a first dose of the post-surgical combination therapy is administered to the subject about 6 weeks following the step of surgically resecting all or a portion of the tumor.

43. The method of any of claims 31-41, wherein the activatable anti-PDL1 antibody and the anti-CTLA-4 antibody components of the post-surgical combination therapy are both administered by intravenous infusion.

44. The method of any of claims 31-43, wherein administering the post-surgical combination therapy to the subject comprises administering the activatable anti-PDL1 antibody component of the post-surgical combination therapy prior to administering the anti-CTLA-4 component of the post-surgical combination therapy.

45. The method of any of claims 31-44, wherein administering the post-surgical combination therapy comprises administering the activatable anti-PDL1 antibody and the anti-CTLA-4 antibody components of the post-surgical combination therapy to the subject on the same day.

46. The method of any of claims 31-45, wherein administering the post-surgical combination therapy comprises administering the activatable anti-PDL1 antibody component of the post-surgical combination therapy by intravenous infusion over a period of 60 minutes.

47. The method of any of claims 31-46, wherein administering the post-surgical combination therapy comprises administering the anti-CTLA-4 antibody component of the post-surgical combination therapy by intravenous infusion over a period of 30 minutes.

48. The method of any of claims 31-47, wherein administering the post-surgical combination therapy comprises administering the anti-CTLA-4 antibody component of the post-surgical combination therapy no sooner than 30 minutes after administering the activatable anti-PDL1 antibody component of the post-surgical combination therapy.

49. The method of any of claims 31-45, wherein administering the post-surgical combination therapy comprises:
(i) administering the activatable anti-PDL1 antibody by intravenous infusion over a period of 60 minutes;
(ii) administering a saline flush; and
(iii) administering the anti-CTLA-4 antibody by intravenous infusion over a period of 30 minutes,
wherein the step of administering the anti-CTLA-4 antibody is carried out no sooner than 30 minutes after completion of the step of administering the activatable anti-PDL1 antibody.

50. The method of any of claims 31-49, wherein the method further comprises administering to the subject one or more doses of a post-surgical monotherapy comprising a dose of the activatable anti-PDL1 antibody following administration of the one or more doses of the post-surgical combination therapy.

51. The method of any of claims 31-49, wherein multiple doses of the post-surgical monotherapy are administered to the subject at a frequency of one dose of the activatable anti-PDL1 antibody per interval of time over a second post-surgical period of time.

52. The method of claim 51, wherein the multiple doses of the post-surgical monotherapy are administered to the subject at a frequency of one dose of the activatable anti-PDL1 antibody every 2 weeks.

53. The method of any of claims 50-52, wherein the post-surgical monotherapy dose of activatable anti-PDL1 antibody is a fixed dose in the range of from 240 mg to 2400 mg.

54. The method of any of claims 50-53, wherein the post-surgical monotherapy dose of activatable anti-PDL1 antibody is a fixed dose of 800 mg.

55. The method of any of claims 50-52, wherein the post-surgical monotherapy dose of activatable anti-PDL1 antibody is a dose in the range of from 0.3 mg/kg to 30 mg/kg.

56. The method of any of claims 51-52, wherein multiple doses of the post-surgical monotherapy are administered to the subject at a frequency of one dose of 800 mg of the activatable anti-PDL1 antibody every 2 weeks following administration of the last dose of the post-surgical combination therapy.

57. The method of any of claims 50-56, wherein a first dose of the post-surgical monotherapy is administered to the subject 3 weeks following the last dose of the post-surgical combination therapy.

58. The method of any of claims 51-57, wherein the second post-surgical period of time is about one year.

59. The method of any of claims 50-58, wherein the post-surgical monotherapy dose of the activatable anti-PDL1 antibody is administered as an intravenous infusion.

60. The method of any of claims 1-59, wherein the anti-CTLA-4 antibody is ipilimumab.

61. The method of any of claims 1-60, wherein the cancer is melanoma.

62. The method of any of claims 1-61, wherein the cancer is resectable Stage III melanoma.

63. An activatable anti-PDL1 antibody for use in the treatment of a cancer, wherein the treatment comprises administering the activatable anti-PDL1 antibody intravenously to a subject in a neoadjuvant combination with an anti-CTLA-4 antibody that is administered intravenously to the subject,
wherein the activatable anti-PDL1 antibody comprises:
(i) an antibody or antigen-binding portion thereof that binds human PDL1 (AB) that comprises:
a heavy chain variable region (VH) comprising a complementarity determining region 1 (CDRH1) that comprises the amino acid sequence of SEQ ID NO:125, a complementarity determining region 2 (CDRH2) that comprises the amino acid sequence of SEQ ID NO:126, and a complementarity determining region 3 (CDRH3) that comprises the amino acid sequence of SEQ ID NO:127, and
a light chain variable region (VL) comprising a light chain complementarity determining region 1 (CDRL1) that comprises the amino acid sequence of SEQ ID NO:128, a light chain complementarity determining region 2 (CDRL2) that comprises the amino acid sequence of SEQ ID NO:129, and a light chain complementarity determining region 3 (CDRL3) that comprises the amino acid sequence of SEQ ID NO:130;
(ii) a cleavable moiety (CM) linked, directly or indirectly, to the AB, wherein the CM is a polypeptide that functions as a substrate for a protease; and
(iii) a masking moiety (MM) linked, directly or indirectly, to the CM; and
(B) an anti-CTLA-4 antibody,
wherein the subject has a solid tumor.

64. The activatable anti-PDL1 antibody of claim 63, wherein the activatable anti-PDL1 antibody component of the neoadjuvant combination is administered to the subject at a fixed dose of 800 mg.

65. The activatable anti-PDL1 antibody of any of claims 63-64, wherein the anti-CTLA-4 antibody component of the neoadjuvant combination is administered to the subject at a dose of less than 3 mg/kg or at a dose selected from the group consisting of 1 mg/kg and 2 mg/kg.

66. An activatable anti-PDL1 antibody for use in the treatment of a cancer, wherein the treatment comprises administering the activatable anti-PDL1 antibody intravenously to a subject at a fixed dose of 800 mg in a neoadjuvant combination with an anti-CTLA-4 antibody that is administered intravenously to the subject at a dose of 1 mg/kg,
wherein the activatable anti-PDL1 antibody comprises:
(i) an antibody or antigen-binding portion thereof that binds human PDL1 (AB) that comprises:
a heavy chain variable region (VH) comprising a complementarity determining region 1 (CDRH1) that comprises the amino acid sequence of SEQ ID NO:125, a complementarity determining region 2 (CDRH2) that comprises the amino acid sequence of SEQ ID NO:126, and a complementarity determining region 3 (CDRH3) that comprises the amino acid sequence of SEQ ID NO:127, and
a light chain variable region (VL) comprising a light chain complementarity determining region 1 (CDRL1) that comprises the amino acid sequence of SEQ ID NO:128, a light chain complementarity determining region 2 (CDRL2) that comprises the amino acid sequence of SEQ ID NO:129, and a light chain complementarity determining region 3 (CDRL3) that comprises the amino acid sequence of SEQ ID NO:130;
(ii) a cleavable moiety (CM) linked, directly or indirectly, to the AB, wherein the CM is a polypeptide that functions as a substrate for a protease; and
(iii) a masking moiety (MM) linked, directly or indirectly, to the CM; and
wherein the subject has a solid tumor.

67. The activatable anti-PDL1 antibody of any of claims 63-66, wherein the MM comprises an amino acid sequence selected from the group consisting of SEQ ID NOs:1-25.

68. The activatable anti-PDL1 antibody any of claims 64-67, wherein the MM comprises the amino acid sequence of SEQ ID NO:7.

69. The activatable anti-PDL1 antibody of any of claims 63-68, wherein the CM comprises an amino acid sequence selected from the group consisting of SEQ ID NOs:26-92.

70. The activatable anti-PDL1 antibody of any of claims 63-69, wherein the CM comprises the amino acid sequence of SEQ ID NO:49.

71. The activatable anti-PDL1 antibody of any of claims 63-70, wherein the activatable anti-PDL1 antibody comprises a heavy chain variable region (VH) comprising the amino acid sequence of SEQ ID NO:118, and a light chain variable region (VL) comprising the amino acid sequence of SEQ ID NO:119.

72. The activatable anti-PDL1 antibody of any of claims 63-70, wherein the activatable anti-PDL1 antibody comprises a light chain and a heavy chain, and wherein the light chain comprises the MM, the CM, and the VL, and wherein the light chain comprises the amino acid sequence of SEQ ID NO:120, and wherein the heavy chain comprises the VH which comprises the amino acid sequence of SEQ ID NO:118.

73. The activatable anti-PDL1 antibody of any of claims 63-70, wherein the activatable anti-PDL1 antibody comprises a light chain and a heavy chain, wherein the light chain comprises the MM, the CM, and the VL, and wherein the light chain comprises the amino acid sequence of SEQ ID NO:121, and wherein the heavy chain comprises the VH which comprises the amino acid sequence of SEQ ID NO:118.

74. An activatable anti-PDL1 antibody for use in the treatment of a cancer, wherein the treatment comprises administering the activatable anti-PDL1 antibody intravenously to a subject at a fixed dose of 800 mg in a neoadjuvant combination with an anti-CTLA-4 antibody that is administered intravenously to the subject at a dose of 1 mg/kg,
wherein the activatable anti-PDL1 antibody comprises a heavy chain comprising the amino acid sequence of SEQ ID NO:122, and a light chain comprising the amino acid sequence selected from the group consisting of SEQ ID NO:123 and SEQ ID NO:124,
wherein the activatable anti-PDL1 antibody comprises an antibody or antigen-binding portion thereof that binds human PDL1 (AB), a cleavable moiety (CM), and a masking moiety (MM), and
wherein the subject has a solid tumor.

75. The activatable anti-PDL1 antibody of claim 74, wherein the light chain comprises the amino acid sequence of SEQ ID NO:123.

76. The activatable anti-PDL1 antibody of claim 74, wherein the light chain comprises the amino acid sequence of SEQ ID NO:124.

77. The activatable anti-PDL1 antibody of any of claims 63-76, wherein the treatment comprises administering multiple doses of the neoadjuvant combination to the subject at a frequency of one dose of the neoadjuvant combination per interval of time.

78. The activatable anti-PDL1 antibody of claim 77, wherein 2 doses of the neoadjuvant combination are administered to the subject at a frequency of one dose per interval of time.

79. The activatable anti-PDL1 antibody of any of claims 77-78, wherein the multiple doses of the neoadjuvant combination are administered to the subject at a frequency of one dose of the neoadjuvant combination every 3 weeks.

80. The activatable anti-PDL1 antibody of any of claims 77-79, wherein each dose of the activatable anti-PDL1 antibody in the multiple doses is the same, and each dose of the anti-CTLA-4 antibody in the multiple doses is the same.

81. The activatable anti-PDL1 antibody of any of claims 63-80, wherein the treatment further comprises surgically resecting all or a portion of the solid tumor after administration of a last dose of the neoadjuvant combination.

82. The activatable anti-PDL1 antibody of claim 81, wherein the step of surgically resecting all or a portion of the solid tumor occurs 6 weeks after administration of a last dose of the neoadjuvant combination.

83. The activatable anti-PDL1 antibody of any of claims 63-82, wherein the activatable anti-PDL1 antibody component of the neoadjuvant combination is administered to the subject prior to administering the anti-CTLA-4 component of the neoadjuvant combination.

84. The activatable anti-PDL1 antibody of any of claims 63-83, wherein the activatable anti-PDL1 antibody component of the neoadjuvant combination is administered by intravenous infusion over a period of 60 minutes.

85. The activatable anti-PDL1 antibody of any of claims 63-84, wherein the anti-CTLA-4 antibody component of the neoadjuvant combination is administered to the subject by intravenous infusion over a period of 30 minutes.

86. The activatable anti-PDL1 antibody of any of claims 63-85, wherein the anti-CTLA-4 antibody component of the neoadjuvant combination is administered to the subject no sooner than 30 minutes after administering the activatable anti-PDL1 antibody component of the neoadjuvant combination.

87. The activatable anti-PDL1 antibody of any of claims 63-83, wherein the treatment comprises:
(i) administering the activatable anti-PDL1 antibody by intravenous infusion over a period of 60 minutes;
(ii) administering a saline flush; and
(iii) administering the anti-CTLA-4 antibody by intravenous infusion over a period of 30 minutes,
wherein the step of administering the anti-CTLA-4 antibody is carried out no sooner than 30 minutes after completion of the step of administering the activatable anti-PDL1 antibody.

88. The activatable anti-PDL1 antibody of any of claims 63-87, wherein the treatment further comprises administering to the subject one or more doses of a post-surgical combination therapy comprising a dose of the activatable anti-PDL1 antibody and a dose of the anti-CTLA-4 antibody.

89. The activatable anti-PDL1 antibody of claim 88, wherein multiple doses of the post-surgical combination therapy are administered to the subject at a frequency of one dose of the post-surgical combination therapy per interval of time over a first post-surgical period of time.

90. The activatable anti-PDL1 antibody of any of claims 88-89, wherein two doses of the post-surgical combination therapy are administered to the subject at a frequency of one dose per interval of time over the first post-surgical period of time.

91. The activatable anti-PDL1 antibody of any of claims 88-90, wherein multiple doses of the post-surgical combination therapy are administered to the subject at a frequency of one dose of the post-surgical combination therapy every 3 weeks.

92. The activatable anti-PDL1 antibody of any of claims 88-91, wherein the activatable anti-PDL1 antibody component of the post-surgical combination therapy is administered to the subject at a fixed dose of 800 mg, and the anti-CTLA-4 antibody component of the post-surgical combination therapy is administered to the subject at a dose of 1 mg/kg.

93. The activatable anti-PDL1 antibody of any of claims 88-92, wherein a first dose of the post-surgical combination therapy is administered to the subject within a time period in the range of from 4 to 8 weeks or in the range of from 5 to 7 weeks following a surgical resection of all or a portion of the tumor.

94. The activatable anti-PDL1 antibody of any of claims 88-93, wherein a first dose of the post-surgical combination therapy is administered to the subject about 6 weeks following a surgical resection of all or a portion of the tumor.

95. The activatable anti-PDL1 antibody of any of claims 88-94, wherein the activatable anti-PDL1 antibody and the anti-CTLA-4 antibody components of the post-surgical combination therapy are both administered by intravenous infusion.

96. The activatable anti-PDL1 antibody of any of claims 88-95, wherein
administering the post-surgical combination therapy to the subject comprises administering the activatable anti-PDL1 antibody component of the post-surgical combination therapy prior to administering the anti-CTLA-4 component of the post-surgical combination therapy.

97. The activatable anti-PDL1 antibody of any of claims 88-96, wherein
administering the post-surgical combination therapy comprises administering the activatable anti-PDL1 antibody component of the post-surgical combination therapy by intravenous infusion over a period of 60 minutes.

98. The activatable anti-PDL1 antibody of any of claims 88-97, wherein administering the post-surgical combination therapy comprises administering the anti-CTLA-4 antibody component of the post-surgical combination therapy by intravenous infusion over a period of 30 minutes.

99. The activatable anti-PDL1 antibody of any of claims 88-98, wherein administering the post-surgical combination therapy comprises administering the anti-CTLA-4 antibody component of the post-surgical combination therapy no sooner than 30 minutes after administering the activatable anti-PDL1 antibody component of the post-surgical combination therapy.

100. The activatable anti-PDL1 antibody of any of claims 88-96, wherein the post-surgical combination therapy comprises:
(i) administering the activatable anti-PDL1 antibody by intravenous infusion over a period of 60 minutes;
(ii) administering a saline flush; and
(iii) administering the anti-CTLA-4 antibody by intravenous infusion over a period of 30 minutes,
wherein the step of administering the anti-CTLA-4 antibody is carried out no sooner than 30 minutes after completion of the step of administering the activatable anti-PDL1 antibody.

101. The activatable anti-PDL1 antibody of any of claims 63-100, wherein the treatment further comprises administering to the subject one or more doses of a post-surgical monotherapy comprising a dose of the activatable anti-PDL1 antibody following administration of the one or more doses of the post-surgical combination therapy.

102. The activatable anti-PDL1 antibody of claim 101, wherein multiple doses of the post-surgical monotherapy are administered to the subject at a frequency of one dose of the activatable anti-PDL1 antibody per interval of time over a second post-surgical period of time.

103. The activatable anti-PDL1 antibody of claim 102, wherein the multiple doses of the post-surgical monotherapy are administered to the subject at a frequency of one dose of the activatable anti-PDL1 antibody every 2 weeks.

104. The activatable anti-PDL1 antibody of claim 102, wherein the post-surgical monotherapy dose is a fixed dose of 800 mg of the activatable anti-PDL1 antibody.

105. The activatable anti-PDL1 antibody of any of claims 101-104, wherein a first dose of the post-surgical monotherapy is administered to the subject 3 weeks following the last dose of the post-surgical combination therapy.

106. The activatable anti-PDL1 antibody of any of claims 101-105, wherein the post-surgical monotherapy dose of the activatable anti-PDL1 antibody is administered as an intravenous infusion.

107. The activatable anti-PDL1 antibody of any of claims 63-106, wherein the anti-CTLA-4 antibody is ipilimumab.

108. The activatable anti-PDL1 antibody of any of claims 63-107, wherein the cancer is melanoma.

109. The activatable anti-PDL1 antibody of any of claims 63-108, wherein the cancer is resectable Stage III melanoma.


SEQUENCE LISTING (130 sequences)

SEQ ID NO: 1 - protein, 15 aa, Artificial Sequence
  Synthetic
YCEVSELFVLPWCMG

SEQ ID NO: 2 - protein, 15 aa, Artificial Sequence
  Synthetic
SCLMHPHYAHDYCYV

SEQ ID NO: 3 - protein, 15 aa, Artificial Sequence
  Synthetic
LCEVLMLLQHPWCMG

SEQ ID NO: 4 - protein, 15 aa, Artificial Sequence
  Synthetic
IACRHFMEQLPFCHH

SEQ ID NO: 5 - protein, 15 aa, Artificial Sequence
  Synthetic
FGPRCGEASTCVPYE

SEQ ID NO: 6 - protein, 15 aa, Artificial Sequence
  Synthetic
ILYCDSWGAGCLTRP

SEQ ID NO: 7 - protein, 15 aa, Artificial Sequence
  Synthetic
GIALCPSHFCQLPQT

SEQ ID NO: 8 - protein, 15 aa, Artificial Sequence
  Synthetic
DGPRCFVSGECSPIG

SEQ ID NO: 9 - protein, 15 aa, Artificial Sequence
  Synthetic
LCYKLDYDDRSYCHI

SEQ ID NO: 10 - protein, 15 aa, Artificial Sequence
  Synthetic
PCHPHPYDARPYCNV

SEQ ID NO: 11 - protein, 15 aa, Artificial Sequence
  Synthetic
PCYWHPFFAYRYCNT

SEQ ID NO: 12 - protein, 15 aa, Artificial Sequence
  Synthetic
VCYYMDWLGRNWCSS

SEQ ID NO: 13 - protein, 15 aa, Artificial Sequence
  Synthetic
LCDLFKLREFPYCMG

SEQ ID NO: 14 - protein, 15 aa, Artificial Sequence
  Synthetic
YLPCHFVPIGACNNK

SEQ ID NO: 15 - protein, 15 aa, Artificial Sequence
  Synthetic
IFCHMGVVVPQCANY

SEQ ID NO: 16 - protein, 15 aa, Artificial Sequence
  Synthetic
ACHPHPYDARPYCNV

SEQ ID NO: 17 - protein, 15 aa, Artificial Sequence
  Synthetic
PCHPAPYDARPYCNV

SEQ ID NO: 18 - protein, 15 aa, Artificial Sequence
  Synthetic
PCHPHAYDARPYCNV

SEQ ID NO: 19 - protein, 15 aa, Artificial Sequence
  Synthetic
PCHPHPADARPYCNV

SEQ ID NO: 20 - protein, 15 aa, Artificial Sequence
  Synthetic
PCHPHPYAARPYCNV

SEQ ID NO: 21 - protein, 15 aa, Artificial Sequence
  Synthetic
PCHPHPYDAAPYCNV

SEQ ID NO: 22 - protein, 15 aa, Artificial Sequence
  Synthetic
PCHPHPYDARPACNV

SEQ ID NO: 23 - protein, 15 aa, Artificial Sequence
  Synthetic
PCHPHPYDARPYCAV

SEQ ID NO: 24 - protein, 15 aa, Artificial Sequence
  Synthetic
PCHAHPYDARPYCNV

SEQ ID NO: 25 - protein, 15 aa, Artificial Sequence
  Synthetic
PCHPHPYDARAYCNV

SEQ ID NO: 26 - protein, 8 aa, Artificial Sequence
  Synthetic
LSGRSDNH

SEQ ID NO: 27 - protein, 8 aa, Artificial Sequence
  Synthetic
TGRGPSWV

SEQ ID NO: 28 - protein, 8 aa, Artificial Sequence
  Synthetic
PLTGRSGG

SEQ ID NO: 29 - protein, 8 aa, Artificial Sequence
  Synthetic
TARGPSFK

SEQ ID NO: 30 - protein, 12 aa, Artificial Sequence
  Synthetic
NTLSGRSENHSG

SEQ ID NO: 31 - protein, 12 aa, Artificial Sequence
  Synthetic
NTLSGRSGNHGS

SEQ ID NO: 32 - protein, 12 aa, Artificial Sequence
  Synthetic
TSTSGRSANPRG

SEQ ID NO: 33 - protein, 8 aa, Artificial Sequence
  Synthetic
TSGRSANP

SEQ ID NO: 34 - protein, 10 aa, Artificial Sequence
  Synthetic
VHMPLGFLGP

SEQ ID NO: 35 - protein, 8 aa, Artificial Sequence
  Synthetic
AVGLLAPP

SEQ ID NO: 36 - protein, 8 aa, Artificial Sequence
  Synthetic
AQNLLGMV

SEQ ID NO: 37 - protein, 8 aa, Artificial Sequence
  Synthetic
QNQALRMA

SEQ ID NO: 38 - protein, 8 aa, Artificial Sequence
  Synthetic
LAAPLGLL

SEQ ID NO: 39 - protein, 8 aa, Artificial Sequence
  Synthetic
STFPFGMF

SEQ ID NO: 40 - protein, 8 aa, Artificial Sequence
  Synthetic
ISSGLLSS

SEQ ID NO: 41 - protein, 8 aa, Artificial Sequence
  Synthetic
PAGLWLDP

SEQ ID NO: 42 - protein, 8 aa, Artificial Sequence
  Synthetic
VAGRSMRP

SEQ ID NO: 43 - protein, 8 aa, Artificial Sequence
  Synthetic
VVPEGRRS

SEQ ID NO: 44 - protein, 8 aa, Artificial Sequence
  Synthetic
ILPRSPAF

SEQ ID NO: 45 - protein, 8 aa, Artificial Sequence
  Synthetic
MVLGRSLL

SEQ ID NO: 46 - protein, 8 aa, Artificial Sequence
  Synthetic
QGRAITFI

SEQ ID NO: 47 - protein, 8 aa, Artificial Sequence
  Synthetic
SPRSIMLA

SEQ ID NO: 48 - protein, 8 aa, Artificial Sequence
  Synthetic
SMLRSMPL

SEQ ID NO: 49 - protein, 13 aa, Artificial Sequence
  Synthetic
ISSGLLSGRSDNH

SEQ ID NO: 50 - protein, 18 aa, Artificial Sequence
  Synthetic
AVGLLAPPGGLSGRSDNH

SEQ ID NO: 51 - protein, 22 aa, Artificial Sequence
  Synthetic
ISSGLLSSGGSGGSLSGRSDNH

SEQ ID NO: 52 - protein, 8 aa, Artificial Sequence
  Synthetic
LSGRSGNH

SEQ ID NO: 53 - protein, 9 aa, Artificial Sequence
  Synthetic
SGRSANPRG

SEQ ID NO: 54 - protein, 8 aa, Artificial Sequence
  Synthetic
LSGRSDDH

SEQ ID NO: 55 - protein, 8 aa, Artificial Sequence
  Synthetic
LSGRSDIH

SEQ ID NO: 56 - protein, 8 aa, Artificial Sequence
  Synthetic
LSGRSDQH

SEQ ID NO: 57 - protein, 8 aa, Artificial Sequence
  Synthetic
LSGRSDTH

SEQ ID NO: 58 - protein, 8 aa, Artificial Sequence
  Synthetic
LSGRSDYH

SEQ ID NO: 59 - protein, 8 aa, Artificial Sequence
  Synthetic
LSGRSDNP

SEQ ID NO: 60 - protein, 8 aa, Artificial Sequence
  Synthetic
LSGRSANP

SEQ ID NO: 61 - protein, 8 aa, Artificial Sequence
  Synthetic
LSGRSANI

SEQ ID NO: 62 - protein, 8 aa, Artificial Sequence
  Synthetic
LSGRSDNI

SEQ ID NO: 63 - protein, 8 aa, Artificial Sequence
  Synthetic
MIAPVAYR

SEQ ID NO: 64 - protein, 8 aa, Artificial Sequence
  Synthetic
RPSPMWAY

SEQ ID NO: 65 - protein, 8 aa, Artificial Sequence
  Synthetic
WATPRPMR

SEQ ID NO: 66 - protein, 8 aa, Artificial Sequence
  Synthetic
FRLLDWQW

SEQ ID NO: 67 - protein, 5 aa, Artificial Sequence
  Synthetic
ISSGL

SEQ ID NO: 68 - protein, 7 aa, Artificial Sequence
  Synthetic
ISSGLLS

SEQ ID NO: 69 - protein, 6 aa, Artificial Sequence
  Synthetic
ISSGLL

SEQ ID NO: 70 - protein, 15 aa, Artificial Sequence
  Synthetic
ISSGLLSGRSANPRG

SEQ ID NO: 71 - protein, 18 aa, Artificial Sequence
  Synthetic
AVGLLAPPTSGRSANPRG

SEQ ID NO: 72 - protein, 17 aa, Artificial Sequence
  Synthetic
AVGLLAPPSGRSANPRG

SEQ ID NO: 73 - protein, 13 aa, Artificial Sequence
  Synthetic
ISSGLLSGRSDDH

SEQ ID NO: 74 - protein, 13 aa, Artificial Sequence
  Synthetic
ISSGLLSGRSDIH

SEQ ID NO: 75 - protein, 13 aa, Artificial Sequence
  Synthetic
ISSGLLSGRSDQH

SEQ ID NO: 76 - protein, 13 aa, Artificial Sequence
  Synthetic
ISSGLLSGRSDTH

SEQ ID NO: 77 - protein, 13 aa, Artificial Sequence
  Synthetic
ISSGLLSGRSDYH

SEQ ID NO: 78 - protein, 13 aa, Artificial Sequence
  Synthetic
ISSGLLSGRSDNP

SEQ ID NO: 79 - protein, 13 aa, Artificial Sequence
  Synthetic
ISSGLLSGRSANP

SEQ ID NO: 80 - protein, 13 aa, Artificial Sequence
  Synthetic
ISSGLLSGRSANI

SEQ ID NO: 81 - protein, 18 aa, Artificial Sequence
  Synthetic
AVGLLAPPGGLSGRSDDH

SEQ ID NO: 82 - protein, 18 aa, Artificial Sequence
  Synthetic
AVGLLAPPGGLSGRSDIH

SEQ ID NO: 83 - protein, 18 aa, Artificial Sequence
  Synthetic
AVGLLAPPGGLSGRSDQH

SEQ ID NO: 84 - protein, 18 aa, Artificial Sequence
  Synthetic
AVGLLAPPGGLSGRSDTH

SEQ ID NO: 85 - protein, 18 aa, Artificial Sequence
  Synthetic
AVGLLAPPGGLSGRSDYH

SEQ ID NO: 86 - protein, 18 aa, Artificial Sequence
  Synthetic
AVGLLAPPGGLSGRSDNP

SEQ ID NO: 87 - protein, 18 aa, Artificial Sequence
  Synthetic
AVGLLAPPGGLSGRSANP

SEQ ID NO: 88 - protein, 18 aa, Artificial Sequence
  Synthetic
AVGLLAPPGGLSGRSANI

SEQ ID NO: 89 - protein, 13 aa, Artificial Sequence
  Synthetic
ISSGLLSGRSDNI

SEQ ID NO: 90 - protein, 18 aa, Artificial Sequence
  Synthetic
AVGLLAPPGGLSGRSDNI

SEQ ID NO: 91 - protein, 19 aa, Artificial Sequence
  Synthetic
GLSGRSDNHGGAVGLLAPP

SEQ ID NO: 92 - protein, 21 aa, Artificial Sequence
  Synthetic
GLSGRSDNHGGVHMPLGFLGP

SEQ ID NO: 93 - protein, 5 aa, Artificial Sequence
  Synthetic
GSGGS

SEQ ID NO: 94 - protein, 4 aa, Artificial Sequence
  Synthetic
GGGS

SEQ ID NO: 95 - protein, 4 aa, Artificial Sequence
  Synthetic
GGSG

SEQ ID NO: 96 - protein, 5 aa, Artificial Sequence
  Synthetic
GGSGG

SEQ ID NO: 97 - protein, 5 aa, Artificial Sequence
  Synthetic
GSGSG

SEQ ID NO: 98 - protein, 5 aa, Artificial Sequence
  Synthetic
GSGGG

SEQ ID NO: 99 - protein, 11 aa, Artificial Sequence
  Synthetic
GSSGGSGGSGG

SEQ ID NO: 100 - protein, 12 aa, Artificial Sequence
  Synthetic
GSSGGSGGSGGS

SEQ ID NO: 101 - protein, 16 aa, Artificial Sequence
  Synthetic
GSSGGSGGSGGSGGGS

SEQ ID NO: 102 - protein, 10 aa, Artificial Sequence
  Synthetic
GSSGGSGGSG

SEQ ID NO: 103 - protein, 11 aa, Artificial Sequence
  Synthetic
GSSGGSGGSGS

SEQ ID NO: 104 - protein, 4 aa, Artificial Sequence
  Synthetic
GGGS

SEQ ID NO: 105 - protein, 5 aa, Artificial Sequence
  Synthetic
GSSGT

SEQ ID NO: 106 - protein, 4 aa, Artificial Sequence
  Synthetic
GSSG

SEQ ID NO: 107 - protein, 13 aa, Artificial Sequence
  Synthetic
GGGSSGGSGGSGG

SEQ ID NO: 108 - protein, 6 aa, Artificial Sequence
  Synthetic
QGQSGS

SEQ ID NO: 109 - protein, 5 aa, Artificial Sequence
  Synthetic
GQSGS

SEQ ID NO: 110 - protein, 4 aa, Artificial Sequence
  Synthetic
QSGS

SEQ ID NO: 111 - protein, 7 aa, Artificial Sequence
  Synthetic
QGQSGQG

SEQ ID NO: 112 - protein, 6 aa, Artificial Sequence
  Synthetic
GQSGQG

SEQ ID NO: 113 - protein, 5 aa, Artificial Sequence
  Synthetic
QSGQG

SEQ ID NO: 114 - protein, 4 aa, Artificial Sequence
  Synthetic
SGQG

SEQ ID NO: 115 - protein, 6 aa, Artificial Sequence
  Synthetic
QGQSGQ

SEQ ID NO: 116 - protein, 5 aa, Artificial Sequence
  Synthetic
GQSGQ

SEQ ID NO: 117 - protein, 4 aa, Artificial Sequence
  Synthetic
QSGQ

SEQ ID NO: 118 - protein, 116 aa, Artificial Sequence
  Synthetic
EVQLLESGGGLVQPGGSLRLSCAASGFTFSSYAMSWVRQAPGKGLEWVSSIWRNGIVTVYADSVKGRFTISRDNSKNTLYLQMNSLRAEDTAVYYCAKWSAAFDYWGQGTLVTVSS

SEQ ID NO: 119 - protein, 108 aa, Artificial Sequence
  Synthetic
DIQMTQSPSSLSASVGDRVTITCRASQSISSYLNWYQQKPGKAPKLLIYAASSLQSGVPSRFSGSGSGTDFTLTISSLQPEDFATYYCQQDNGYPSTFGGGTKVEIKR

SEQ ID NO: 120 - protein, 152 aa, Artificial Sequence
  Synthetic
GIALCPSHFCQLPQTGGGSSGGSGGSGGISSGLLSGRSDNHGGSDIQMTQSPSSLSASVGDRVTITCRASQSISSYLNWYQQKPGKAPKLLIYAASSLQSGVPSRFSGSGSGTDFTLTISSLQPEDFATYYCQQDNGYPSTFGGGTKVEIKR

SEQ ID NO: 121 - protein, 158 aa, Artificial Sequence
  Synthetic
QGQSGSGIALCPSHFCQLPQTGGGSSGGSGGSGGISSGLLSGRSDNHGGSDIQMTQSPSSLSASVGDRVTITCRASQSISSYLNWYQQKPGKAPKLLIYAASSLQSGVPSRFSGSGSGTDFTLTISSLQPEDFATYYCQQDNGYPSTFGGGTKVEIKR

SEQ ID NO: 122 - protein, 442 aa, Artificial Sequence
  Synthetic
EVQLLESGGGLVQPGGSLRLSCAASGFTFSSYAMSWVRQAPGKGLEWVSSIWRNGIVTVYADSVKGRFTISRDNSKNTLYLQMNSLRAEDTAVYYCAKWSAAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 123 - protein, 258 aa, Artificial Sequence
  Synthetic
GIALCPSHFCQLPQTGGGSSGGSGGSGGISSGLLSGRSDNHGGSDIQMTQSPSSLSASVGDRVTITCRASQSISSYLNWYQQKPGKAPKLLIYAASSLQSGVPSRFSGSGSGTDFTLTISSLQPEDFATYYCQQDNGYPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 264 aa, Artificial Sequence
  Synthetic
QGQSGSGIALCPSHFCQLPQTGGGSSGGSGGSGGISSGLLSGRSDNHGGSDIQMTQSPSSLSASVGDRVTITCRASQSISSYLNWYQQKPGKAPKLLIYAASSLQSGVPSRFSGSGSGTDFTLTISSLQPEDFATYYCQQDNGYPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 5 aa, Artificial Sequence
  Synthetic
SYAMS

SEQ ID NO: 126 - protein, 15 aa, Artificial Sequence
  Synthetic
SSIWRNGIVTVYADS

SEQ ID NO: 127 - protein, 7 aa, Artificial Sequence
  Synthetic
WSAAFDY

SEQ ID NO: 128 - protein, 11 aa, Artificial Sequence
  Synthetic
RASQSISSYLN

SEQ ID NO: 129 - protein, 7 aa, Artificial Sequence
  Synthetic
AASSLQS

SEQ ID NO: 130 - protein, 7 aa, Artificial Sequence
  Synthetic
DNGYPST